(19) United States
(12) Patent Application Publication
(10) Pub. No.: US 2025/0114477 A1
(43) Pub. Date: Apr. 10, 2025

(54) POLYNUCLEOTIDE COMPOSITIONS, RELATED FORMULATIONS, AND METHODS OF USE THEREOF

(71) Applicant: ReCode Therapeutics, Inc., Menlo Park, CA (US)

(72) Inventors: Brandon A. Wustman, Menlo Park, CA (US); David J. Lockhart, Menlo Park, CA (US); Mirko Hennig, Menlo Park, CA (US); Daniella Ishimaru, Menlo Park, CA (US); Rumpa Bhattacharjee, Menlo Park, CA (US)

(21) Appl. No.: 18/283,143

(22) PCT Filed: Mar. 21, 2022
(86) PCT No.: PCT/US2022/021196

§ 371 Date: Sep. 20, 2023

Related U.S. Application Data
(60) Provisional application No. 63/164,518, filed on Mar. 22, 2021.
(60) Provisional application No. 63/229,496, filed on Aug. 4, 2021.

(51) Int. Cl. A61K 48/00; A61K 38/17; A61P 11/00

(52) U.S. Cl. CPC A61K 48/0033; A61K 38/1709; A61P 11/00

(57) ABSTRACT

Compositions of polynucleotide(s) are disclosed. A polynucleotide may encode for a polypeptide, protein, or functional fragment thereof associated with primary ciliary dyskinesia (PCD), such as dynein axonemal intermediate chain 1 (DNAI1). Pharmaceutical compositions, kits, and methods for treating a disease or condition associated with cilia maintenance and function, and impaired function of the axoneme are also disclosed. The polynucleotide may be assembled with a lipid composition for delivery to an organ, such as the lung, of a subject. The lipid composition may comprise an ionizable cationic lipid. The polynucleotide can be expressed within cells of the organ of the subject.

### CROSS-REFERENCE

This application claims the benefit of is a U.S. national stage application under 35 U.S.C. 371 of International Patent Application No. PCT/US2022/021196 filed on Mar. 21, 2022, which claims priority to U.S. Provisional Patent Application No. 63/164,518, filed on Mar. 22, 2021, and U.S. Provisional Application Patent No. 63/229,496, filed Aug. 4, 2021, the disclosure of which is incorporated herein by reference in their entirety, including any drawings, each of which is incorporated by reference herein in its entirety.

### BACKGROUND

Nucleic acids, such as messenger ribonucleic acid (mRNA) may be used by cells to express proteins and polypeptides. Some cells may be deficient in a certain protein or nucleic acid and result in disease states. A cell can also take up and translate an exogenous RNA, but many factors influence efficient uptake and translation. For instance, the immune system recognizes many exogenous RNAs as foreign and triggers a response that is aimed at inactivating the RNAs.

### SEQUENCE LISTING

The instant application contains a Sequence Listing, which has been submitted electronically in ASCII format and is hereby incorporated by reference in its entirety. Said ASCII copy, created on or about Feb. 2, 2024, is named 061529-715N01US_ST25.txt and is 13.4 KB in size.

### SUMMARY

Provided here are composition and methods for delivery of nucleic acids. Nucleic acids may be used as a therapeutic. In particular, mRNA may be delivered to a cell of a subject. Upon delivery of a nucleic acid to a cell, the nucleic acid may be used to synthesize a polypeptide. In the case of a cell or subject with a disease or disorder, the nucleic acid may be effective at acting as a therapeutic by increasing the expression of a polypeptide. In cases, where a disorder or disease is caused or correlated to aberrant expression or activity of polypeptide, the increased in expression of the polypeptide may be beneficial. However, the cells may have limited uptake of exogenous nucleic acids and the delivery of the nucleic acids may benefit from compositions that allow for increase uptake of a nucleic acid.

Additionally, therapeutic agents such as proteins and small molecule therapeutic agents could benefit from organ specific delivery. Many different types of compounds such as chemotherapeutic agents exhibit significant cytotoxicity. If these compounds could be better directed towards delivery to the desired organs, then fewer off target effects will be seen.

In an aspect, the present disclosure provides a pharmaceutical composition comprising a polynucleotide assembled with a lipid composition, wherein: the polynucleotide (1) encodes a dynein axonemal intermediate chain 1 (DNAI1) protein and (2) comprises a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15; and the lipid composition comprises a cationic lipid having a structural formula (I′):

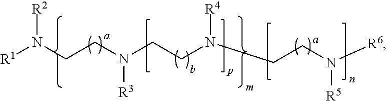

wherein: p is 1 or 2; a is 1 and b is 2, 3, or 4; or, alternatively, b is 1 and a is 2, 3, or 4; m is 1 and n is 1; or, alternatively, m is 2 and n is 0; or, alternatively, m is 2 and n is 1; and R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand; provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and wherein one or more of the nitrogen atoms indicated in formula (I′) may be protonated to provide a cationic lipid.

In some embodiment, a is 1. In some embodiments, b is 2. In some embodiments, m is 1. In some embodiments, R¹, R², R³, R⁴, R⁵, and R⁶ are each independently H or —CH₂CH(OH)R⁷. In some embodiments, R¹, R², R³, R⁴, R⁵, and R⁶ are each independently H or

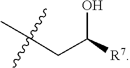

In some embodiments, R¹, R², R³, R⁴, R⁵, and R⁶ are each independently H or

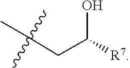

In some embodiments, R⁷ is C₃-C₁₈ alkyl (e.g., C₆-C₁₂ alkyl).

In some embodiments, the cationic lipid is 13,16,20-tris(2-hydroxydodecyl)-13,16,20,23-tetraazapentatricontane-11,25-diol:

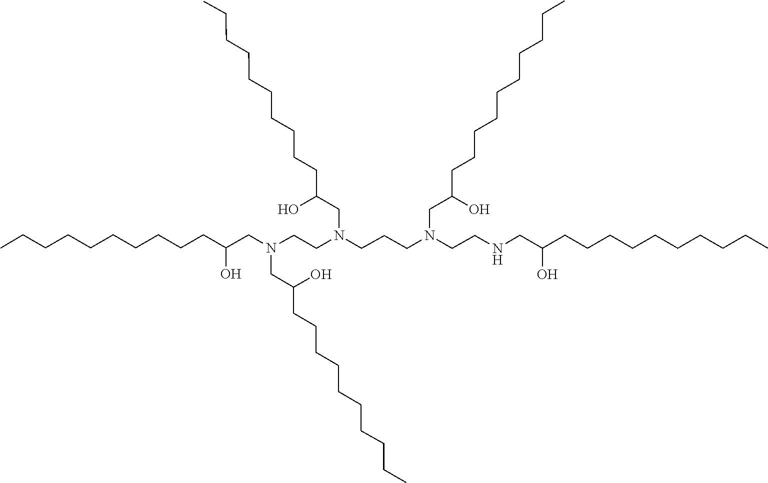 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid is (11R,25R)-13,16,20-tris((R)-2-hydroxydodecyl)-13,16,20,23-tetraazapentatricontane-11,25-diol:

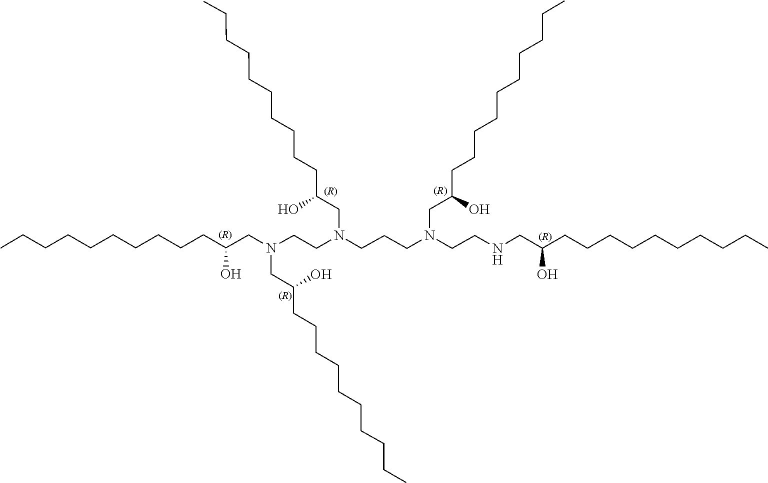

In some embodiments, the lipid composition comprises the cationic lipid at a molar percentage of no more than 50%. In some embodiments, the lipid composition further comprises a phospholipid (e.g., at a molar percentage of at least about 5%). In some embodiments, the lipid composition further comprises a steroid or steroid derivative. In some embodiments, the lipid composition further comprises a polymer-conjugated lipid (e.g., poly(ethylene glycol) (PEG)-conjugated lipid).

In some embodiments, the nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotide residues 1 to 1,000) of SEQ ID NO: 15. In some embodiments, the nucleic acid sequence has 100% sequence identity to a sequence over at least 1,000 bases (e.g., nucleotide residues 1 to 1,000) of SEQ ID NO: 15. In some embodiments, at least 90%, 95%, or 97% nucleotides replacing uridine within the polynucleotide are nucleotide analogues. In some embodiments, fewer than 15% of nucleotides within the polynucleotide are nucleotide analogues. In some embodiments, the polynucleotide comprises 1-methylpseudouridine. In some embodiments, the nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NO: 16. In some embodiments, the nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NO: 16. In some embodiments, the nucleic acid sequence comprises fewer codon types encoding an amino acid as compared to a corresponding wild-type sequence selected from SEQ ID NO: 16. In some embodiments, at least one type of an isoleucine-encoding codons in the corresponding wild-type sequence is substituted with a synonymous codon type in the nucleic acid sequence. In some embodiments, at least one type of a valine-encoding codons in the corresponding wild-type sequence is substituted with a synonymous codon type in the nucleic acid sequence. In some embodiments, at least one type of an alanine-encoding codons in the corresponding wild-type sequence is substituted with a synonymous codon type in the nucleic acid sequence. In some embodiments, at least one type of a glycine-encoding codons in the corresponding wild-type sequence is substituted with a synonymous codon type in the nucleic acid sequence. In some embodiments, at least one type of a proline-encoding codons in the corresponding wild-type sequence is substituted with a synonymous codon type in the nucleic acid sequence. In some embodiments, at least one type of a threonine-encoding codons in the corresponding wild-type sequence is substituted with a synonymous codon type in the nucleic acid sequence. In some embodiments, at least one type of a leucine-encoding codons in the corresponding wild-type sequence is substituted with a synonymous codon type in the nucleic acid sequence. In some embodiments, at least one type of an arginine-encoding codons in the corresponding wild-type sequence is substituted with a synonymous codon type in the nucleic acid sequence. In some embodiments, at least one type of a serine-encoding codons in the corresponding wild-type sequence is substituted with a synonymous codon type in the nucleic acid sequence.

In some embodiments, the pharmaceutical composition comprises an excipient. In some embodiments, polynucleotide is present in the pharmaceutical composition at a concentration of no more than 1 mg/mL. In some embodiments, a molar ratio of nitrogen in the lipid composition to phosphate in the polynucleotide (N/P ratio) is no more than about 20:1. In some embodiments, the N/P ratio is from about 5:1 to about 20:1. In some embodiments, a molar ratio of the polynucleotide to total lipids of the lipid composition is no more than about 1:1, 1:10, 1:50, or 1:100. In some embodiments, at least about 85% of the polynucleotide is encapsulated in particles of the lipid compositions. In some embodiments, the lipid composition comprises particles characterized by a (e.g., average) size of 100 nanometers (n) or less. In some embodiments, the lipid composition comprises a plurality of particles characterized by a polydispersity index (PDI) of no more than about 0.2. In some embodiments, the lipid composition comprises a plurality of particles characterized by a negative zeta potential of −5, −4, or −3 millivolts (mv) or a lower negative number.

In some embodiments, the pharmaceutical formulation is formulated for inhalation. In some embodiments, the pharmaceutical composition is an (e.g., inhalable) aerosol composition. In some embodiments, the aerosol composition is generated by a (e.g., at a nebulization rate from 0.2 milliliter (mL) per minute (mL/min) to 1 mL/min). In some embodiments, the aerosol composition has a (e.g., median, or average) droplet size from 1 micron (μm) to 10 μm. In some embodiments, the aerosol composition is generated by a nebulizer at a nebulization rate of no more than 70 mL/minute. In some embodiments, the aerosol composition is generated by a nebulizer at a nebulization rate of no more than 50 mL/minute. In some embodiments, the aerosol composition is generated by a nebulizer at a nebulization rate of no more than 30 mL/minute.

In some embodiments, the aerosol composition has an average droplet size from about to about 0.5 micron (μm) to about 10 μm. In some embodiments, the aerosol composition has an average droplet size from about to about 0.5 micron (μm) to about 10 μm. In some embodiments, the aerosol composition has an average droplet size from about to about 1 micron (μm) to about 10 μm. In some embodiments, the aerosol composition has an average droplet size from about to about 0.5 micron (μm) to about 5 μm. In some embodiments, the droplet size varies less than about 50% for a duration of about 24 hours under a storage condition. In some embodiments, droplets of the aerosol composition are characterized a polydispersity index (PDI) of no more than about 0.5. In some embodiments, the aerosol droplets are generated by a nebulizer at a nebulization rate of no more than 70 mL/minute. In some embodiments, the aerosol droplets have a mass median aerodynamic diameter (MMAD) from about 0.5 micron (μm) to about 10 μm. In some embodiments, the droplet size varies less than about 50% for a duration of about 24 hours under a storage condition. In some embodiments, droplets of said aerosol composition are characterized by a geometric standard deviation (GSD) of no more than about 3.

In another aspect, the present disclosure provides a method for treating a subject having or suspected of having primary ciliary dyskinesia (PCD), comprising administering to the subject a pharmaceutical composition comprising a heterologous polynucleotide assembled with a lipid composition, which heterologous polynucleotide encodes a dynein axonemal intermediate chain 1 (DNAI1) protein, thereby resulting in a heterologous expression of the DNAI1 protein within cells of the subject, wherein the heterologous polynucleotide comprises a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15; and the lipid composition comprises a cationic lipid having a structural formula (I′):

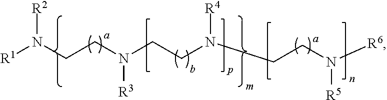

wherein: p is 1 or 2; a is 1 and b is 2, 3, or 4; or, alternatively, b is 1 and a is 2, 3, or 4; m is 1 and n is 1; or, alternatively, m is 2 and n is 0; or, alternatively, m is 2 and n is 1; and R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand; provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and wherein one or more of the nitrogen atoms indicated in formula (I′) may be protonated to provide a cationic lipid.

In some embodiment, a is 1. In some embodiments, b is 2. In some embodiments, m is 1. In some embodiments, R¹, R², R³, R⁴, R⁵, and R⁶ are each independently H or —CH₂CH(OH)R⁷. In some embodiments, R¹, R², R³, R⁴, R⁵, and R⁶ are each independently H or

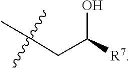

In some embodiments, R¹, R², R³, R⁴, R⁵, and R⁶ are each independently H or

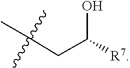

In some embodiments, R⁷ is C₃-C₁₈ alkyl (e.g., C₆-C₁₂ alkyl).

In some embodiments, the cationic lipid is 13,16,20-tris(2-hydroxydodecyl)-13,16,20,23-tetraazapentatricontane-11,25-diol. In some embodiments, the cationic lipid is (11R,25R)-13,16,20-tris((R)-2-hydroxydodecyl)-13,16,20,23-tetraazapentatricontane-11,25-diol. In some embodiments, the administrating comprises administering to a lung by nebulization. In some embodiments, the subject is determined to exhibit an aberrant expression or activity of DNAI1 gene or protein. In some embodiments, the subject is a human. In some embodiments, the cells are in a lung of the subject. In some embodiments, the cells are ciliated cells. In some embodiments, the cells are undifferentiated. In some embodiments, the cells are differentiated. In some embodiments, the ciliated cells are ciliated epithelial cells (e.g., ciliated airway epithelial cells). In some embodiments, the ciliated epithelial cells are undifferentiated. In some embodiments, the ciliated epithelial cells are differentiated.

In another aspect, the present disclosure provides an aerosol composition comprising a pharmaceutical composition described elsewhere herein.

In another aspect, the present disclosure provides a method for treating a subject having or suspected of having primary ciliary dyskinesia (PCD), comprising administering to the subject a pharmaceutical composition described elsewhere herein.

In another aspect, the present disclosure provides a method for enhancing an expression or activity of dynein axonemal intermediate chain 1 (DNAI1) protein in a (e.g., lung) cell, the method comprising: contacting said (e.g., lung) cell with a composition comprising a polynucleotide that encodes a DNAI1 protein (such as described herein), thereby providing a(n) (e.g., therapeutically) effective amount or activity of a functional variant (e.g., wild-type form) of DNAI1 protein in said (e.g., lung) cell. In some embodiments, the method provides a(n) (e.g., therapeutically) effective amount or activity of said functional variant (e.g., wild-type form) of DNAI1 protein in said (e.g., lung) cell at least about 6 hours after said contacting. The contacting may be in vivo. The contacting may be ex vivo. The contacting may be in vitro. In some embodiments, mucus is present in said contacting. In some embodiments, the (e.g., lung) cell is in a ciliary axoneme. In some embodiments, the (e.g., lung) cell is an airway epithelial cell (e.g., a bronchial epithelial cell). In some embodiments, the (e.g., lung) cell exhibits a mutation in DNAI1 gene or transcript. In some embodiments, said contacting comprises contacting a plurality of (e.g., lung) cells that comprises said (e.g., lung) cell.

In some embodiments, said contacting is repeated. In some embodiments, said repeated contacting is at least once a week, at least twice a week, or at least three times a week. In some embodiments, at least one contacting steps of said repeated contacting is followed by a treatment holiday. In some embodiments, the said repeated contacting is characterized by a duration of at least 1, 2, or 3 week(s). In some embodiments, mucus is present in one or more contacting steps of said repeated contacting.

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, or 7 days) after said contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The contacting may be ex vivo or in vitro. The change in the ciliary beat activity may include one or more of the following: an increase or decrease in beat frequency that approaches a normal beat frequency (9-17 Hz), improved synchronization, and an increase in area (e.g., %) with ciliary activity.

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, 7, 8, 9, 10, 11, 12, 13, or 14 days) after a contacting of said repeated contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The repeated contacting(s) may be ex vivo or in vitro. The change in the ciliary beat activity may include one or more of the following: an increase or decrease in beat frequency that approaches a normal beat frequency (9-17 Hz), improved synchronization, and an increase in area (e.g., %) with ciliary activity.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows the chemical structures of lipids.

FIG. 2 shows the chemical structures of dendrimer lipids.

FIGS. 3A and 3B illustrate cell types and expression levels of a delivered mRNA using different compositions of LNP.

FIG. 4 shows images using in vivo imaging of bioluminescence of a mouse after inhaled aerosol delivery of a Luc mRNA/LNP using multiple compositions of LNP.

FIG. 5 shows a chart regarding cell toxicity of various LNP compositions in human bronchial epithelial (hBE) cells.

FIG. 6 shows the stability and general characteristics of various LNP compositions.

FIG. 7 shows a chart of tissue specific radiance over time in a mouse of a LNP composition (“Lung-SORT”; 5A2-SC8 DOTAP).

FIG. 8 shows images of tissue specific radiance over time in a mouse of a LNP composition (“Lung-SORT”; 5A2-SC8 DOTAP).

FIGS. 9A-9B illustrate observed ciliary activity in mouse cells following treatment with repeated doses of an LNP composition.

FIG. 10 shows the chemical structure of 13,16,20-tris(2-hydroxydodecyl)-13,16,20,23-tetraazapentatricontane-11,25-diol.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “disease,” as used herein, generally refers to an abnormal physiological condition that affects part or all of a subject, such as an illness (e.g., primary ciliary dyskinesia) or another abnormality that causes defects in the action of cilia in, for example, the lining the respiratory tract (lower and upper, sinuses, Eustachian tube, middle ear), in a variety of lung cells, in the fallopian tube, or flagella of sperm cells.

The term “polynucleotide” or “nucleic acid” as used herein generally refers to a polymeric form of nucleotides of any length, either ribonucleotides or deoxyribonucleotides, that comprise purine and pyrimidine bases, purine and pyrimidine analogues, chemically or biochemically modified, natural or non-natural, or derivatized nucleotide bases. Polynucleotides include sequences of deoxyribonucleic acid (DNA), ribonucleic acid (RNA), or DNA copies of ribonucleic acid (cDNA), all of which can be recombinantly produced, artificially synthesized, or isolated and purified from natural sources. The polynucleotides and nucleic acids may exist as single-stranded or double-stranded. The backbone of the polynucleotide can comprise sugars and phosphate groups, as may typically be found in RNA or DNA, or analogues or substituted sugar or phosphate groups. A polynucleotide may comprise naturally occurring or non-naturally occurring nucleotides, such as methylated nucleotides and nucleotide analogues (or analogs).

The term “polyribonucleotide,” as used herein, generally refers to polynucleotide polymers that comprise ribonucleic acids. The term also refers to polynucleotide polymers that comprise chemically modified ribonucleotides. A polyribonucleotide can be formed of D-ribose sugars, which can be found in nature.

The term “polypeptides,” as used herein, generally refers to polymer chains comprised of amino acid residue monomers which are joined together through amide bonds (peptide bonds). A polypeptide can be a chain of at least three amino acids, a protein, a recombinant protein, an antigen, an epitope, an enzyme, a receptor, or a structure analogue or combinations thereof. As used herein, the abbreviations for the L-enantiomeric amino acids that form a polypeptide are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). X or Xaa can indicate any amino acid.

The term “engineered,” as used herein, generally refers to polynucleotides, vectors, and nucleic acid constructs that have been genetically designed and manipulated to provide a polynucleotide intracellularly. An engineered polynucleotide can be partially or fully synthesized in vitro. An engineered polynucleotide can also be cloned. An engineered polyribonucleotide can contain one or more base or sugar analogues, such as ribonucleotides not naturally-found in messenger RNAs. An engineered polyribonucleotide can contain nucleotide analogues that exist in transfer RNAs (tRNAs), ribosomal RNAs (rRNAs), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA.

### Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

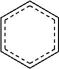

includes

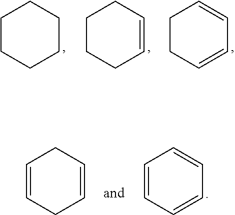

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

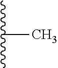

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

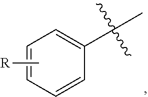

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

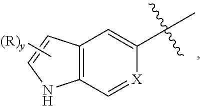

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ^{t}Bu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

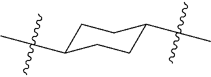 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

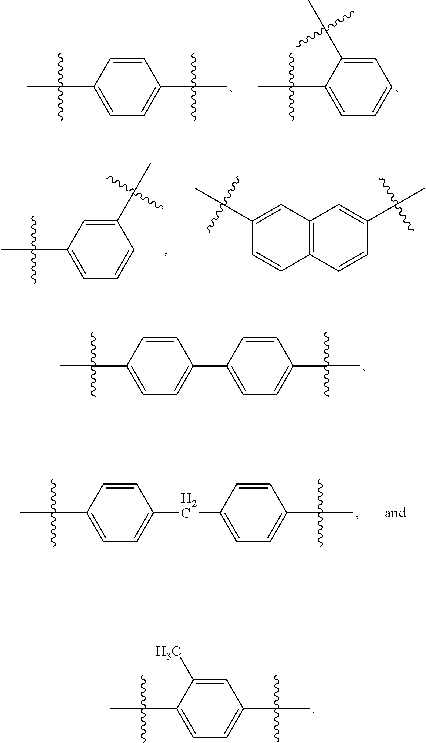

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

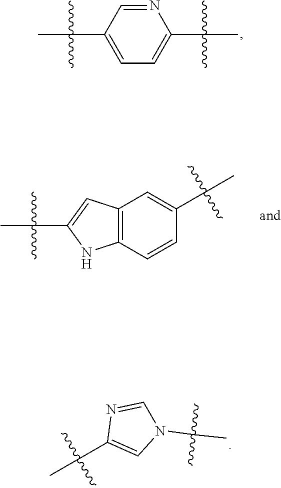 [SMILES: *c1ccc(*)nc1 ; *c1ccc2[nH]c(*)cc2c1 ; *c1cn(*)cn1]

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

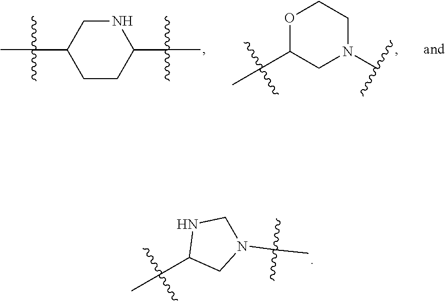 [SMILES: *C1CCC(*)NC1 ; *C1CN(*)CCO1 ; *C1CN(*)CN1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), —OC(CH₃)₃(tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±3%, in another example ±1%, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Examples of lipid compositions include suspensions, emulsions, and vesicular compositions.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “molar percentage” or “molar %” as used herein in connection with lipid composition(s) generally refers to the molar proportion of that component lipid relative to compared to all lipids formulated or present in the lipid composition.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### Primary Ciliary Dyskinesia (PCD)

The present disclosure provides, in some embodiments, compositions and methods for the treatment of conditions associated with cilia maintenance and function, with nucleic acids encoding a protein or protein fragment(s). Numerous eukaryotic cells carry appendages, which are often referred to as cilia or flagella, whose inner core comprises a cytoskeletal structure called the axoneme. The axoneme can function as the skeleton of cellular cytoskeletal structures, both giving support to the structure and, in some instances, causing it to bend. Usually, the internal structure of the axoneme is common to both cilia and flagella. Cilia are often found in the linings of the airway, the reproductive system, and other organs and tissues. Flagella are tail-like structures that, similarly to cilia, can propel cells forward, such as sperm cells.

Without properly functioning cilia in the airway, bacteria can remain in the respiratory tract and cause infection. In the respiratory tract, cilia move back and forth in a coordinated way to move mucus towards the throat. This movement of mucus helps to eliminate fluid, bacteria, and particles from the lungs. Many infants afflicted with cilia and flagella malfunction experience breathing problems at birth, which suggests that cilia play an important role in clearing fetal fluid from the lungs. Beginning in early childhood, subjects afflicted with cilia malfunction can develop frequent respiratory tract infections.

Primary ciliary dyskinesia is a condition characterized by chronic respiratory tract infections, abnormally positioned internal organs, and the inability to have children (infertility). The signs and symptoms of this condition are caused by abnormal cilia and flagella. Subjects afflicted with primary ciliary dyskinesia often have year-round nasal congestion and a chronic cough. Chronic respiratory tract infections can result in a condition called bronchiectasis, which damages the passages, called bronchi, leading from the windpipe to the lungs and can cause life-threatening breathing problems.

The methods, constructs, and compositions of this disclosure provide a method to treat primary ciliary dyskinesia (PCD), also known as immotile ciliary syndrome or Kartagener syndrome. PCD is typically considered to be a rare, ciliopathic, autosomal recessive genetic disorder that often causes defects in the action of cilia lining the respiratory tract (lower and upper, sinuses, Eustachian tube, middle ear) and fallopian tube, as well as in the flagella of sperm cells.

Some individuals with primary ciliary dyskinesia have abnormally placed organs within their chest and abdomen. These abnormalities arise early in embryonic development when the differences between the left and right sides of the body are established. About 50 percent of people with primary ciliary dyskinesia have a mirror-image reversal of their internal organs (situs inversus totalis). For example, in these individuals the heart is on the right side of the body instead of on the left. When someone afflicted with primary ciliary dyskinesia has situs inversus totalis, they are often said to have Kartagener syndrome.

Approximately 12 percent of people with primary ciliary dyskinesia have a condition known as heterotaxy syndrome or situs ambiguus, which is characterized by abnormalities of the heart, liver, intestines, or spleen. These organs may be structurally abnormal or improperly positioned. In addition, affected individuals may lack a spleen (asplenia) or have multiple spleens (polysplenia). Heterotaxy syndrome results from problems establishing the left and right sides of the body during embryonic development. The severity of heterotaxy varies widely among affected individuals.

Primary ciliary dyskinesia can also lead to infertility. Vigorous movements of the flagella are can be needed to propel the sperm cells forward to the female egg cell. Because the sperm of subjects afflicted with primary ciliary dyskinesia does not move properly, males with primary ciliary dyskinesia are usually unable to father children. Infertility occurs in some affected females and it is usually associated with abnormal cilia in the fallopian tubes.

Another feature of primary ciliary dyskinesia is recurrent ear infections (otitis media), especially in young children. Otitis media can lead to permanent hearing loss if untreated. The ear infections are likely related to abnormal cilia within the inner ear.

Rarely, individuals with primary ciliary dyskinesia have an accumulation of fluid in the brain (hydrocephalus), likely due to abnormal cilia in the brain.

At least 21 mutations in the DNAI1 gene have been found to cause primary ciliary dyskinesia, which is a condition characterized by respiratory tract infections, abnormal organ placement, and an inability to have children (infertility). DNAI1 gene mutations result in an absent or abnormal intermediate chain 1. Without a normal version of this subunit, the ODAs cannot form properly and may be shortened or absent. As a result, cilia cannot produce the force needed to bend back and forth. Defective cilia lead to the features of primary ciliary dyskinesia. In some cases, the disclosure provides a nucleic acid that is engineered to replace or to supplement the function of the endogenous DNAI1 protein comprising the IVS1+2_3insT (219+3insT) mutation. In some cases, the disclosure provides a nucleic acid that is engineered to replace or to supplement the function of the endogenous DNAI1 protein comprising the A538T mutation, the second most common.

### Compositions

### Polynucleotides

Provided herein, in some embodiments, is a (e.g., pharmaceutical) composition that comprises a polynucleotide (e.g., comprising a particular sequence that encodes DNAI1). The polynucleotide may comprise a nucleic acid sequence having sequence identity to sequences listed elsewhere herein. The polynucleotide may comprise a nucleic acid sequence having sequence identity to a portion of sequences listed elsewhere herein. For example, the polynucleotide may comprise a nucleic acid sequence having sequence identity to SEQ ID: NO 15. The polynucleotide may comprise a nucleic acid sequence having at a least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence disclosed elsewhere herein. In some embodiments, the nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a fragment (e.g., over at least 500, 600, 700, 800, 900, or 1,000 bases) of SEQ ID NO: 15. The some embodiments, the nucleic acid sequence has 100% sequence identity to a sequence disclosed elsewhere herein. In some embodiments, the nucleic acid has 100% sequence identity to a fragment (e.g., over at least 500, 600, 700, 800, 900, or 1,000 bases) of SEQ ID NO: 15. The polynucleotide may comprise a nucleic acid sequence having at a least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence disclosed elsewhere herein. In some embodiments, the nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotides 1 to 1,000) of SEQ ID NO: 15. The some embodiments, the nucleic acid sequence has 100% sequence identity to a sequence disclosed elsewhere herein. In some embodiments, the nucleic acid has 100% sequence identity to a sequence over at least 1,000 bases of SEQ ID NO: 15. Polynucleotides described elsewhere herein may be DNA or RNA. The sequences disclosed throughout the specification may have a uridine (U) substituted at any location that a thymidine (T) is present. The disclosure recognizes that a sequence; disclosed herein of DNA may be used to generate a corresponding RNA sequence in which instances of thymidine have been replaced with uridine. As such the sequences described herein are not limited to thymidine containing sequence, and the corresponding uridine sequences are also contemplated herein.

The polynucleotide may comprise nucleotide analogues. In some embodiments, the nucleotide analogues replace uridines in a sequence. For example, a sequence using standard nucleotides (A, C, U, T, G) may comprises a uridine at a particular position in a sequence. A sequence may instead have a nucleotide analogue in place of the uridine. The nucleotide analogue may have structure that may still be recognized by the cellular translation machinery such that the polynucleotide comprising a nucleotide analogues may still be translated. The nucleotide analogue may be recognized as synonymous with a standard nucleotide. For example, the nucleotide analogue may be recognized as synonymous with uridine and the resulting translation product is generated as if the nucleotide analogue is a uridine. In some embodiments, at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% of nucleotides replacing uridine within the polynucleotide are nucleotide analogues. In some embodiments, fewer than 15% of nucleotides within the polynucleotide are nucleotide analogues. In some fewer than 30% of the nucleotides are nucleotide analogues. In other cases, fewer than 27.5%, fewer than 25%, fewer than 22.5%, fewer than 20%, fewer than 17.5%, fewer than 15%, fewer than 12.5%, fewer than 10%, fewer than 7.5%, fewer than 5%, or fewer than 2.5% of the nucleotides are nucleotide analogues.

In some embodiments, the nucleotide analogue is a purine or pyrimidine analogue. In some cases, a polyribonucleotide of the disclosure comprises a modified pyrimidine, such as a modified uridine. A nucleotide analogue may be a pseudouridine (Ψ). A nucleotide analogue may be a methylpseudouridine. A nucleotide analogue may be a 1-methylpseudouridine (m¹Ψ). In some embodiments, the polynucleotide comprises a 1-methylpseudouridine. In some cases, a uridine analogue is selected from pseudouridine 1-methylpseudouridine, 2-thiouridine (s²U), 5-methyluridine (m⁵U), 5-methoxyuridine (mo⁵U), 4-thiouridine (s⁴U), 5-bromouridine (Br⁵U), 2′O-methyluridine (U2′m), 2′-amino-2′-deoxyuridine (U2′NH₂), 2′-azido-2′-deoxyuridine (U2′N₃), and 2′-fluoro-2′-deoxyuridine (U2′F).

A polyribonucleotide can have the same or a mixture of different nucleotide analogues or modified nucleotides. The nucleotide analogues or modified nucleotides can have structural changes that are naturally or not naturally occurring in messenger RNA. A mixture of various analogues or modified nucleotides can be used. For example, one or more analogues within a polynucleotide can have natural modifications, while another part has modifications that are not naturally found in mRNA. Additionally, some analogues or modified ribonucleotides can have a base modification, while other modified ribonucleotides have a sugar modification. In the same way, it is possible that all modifications are base modifications or all modifications are sugar modifications or any suitable mixture thereof.

A nucleotide analogue or modified nucleotide can be selected from the group comprising pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some cases, at least about 5% of the nucleic acid construct(s), a vector(s), engineered polyribonucleotide(s), or compositions includes non-naturally occurring (e.g., modified, analogues, or engineered) uridine, adenosine, guanine, or cytosine, such as the nucleotides described herein. In some cases, 100% of the modified nucleotides in the composition are either 1-methylpseudouridine or pseudouridine. In some cases, at least about 10%, 15%, 20%, 25%, 30%, 35%, 40%, 45%, 50%, 55%, 60%, 65%, 70%, 75%, 80%, 85%, 90%, 95% of the nucleic acid construct(s), a vector(s), engineered polyribonucleotide(s), or compositions includes non-naturally occurring uracil, adenine, guanine, or cytosine. In some cases, at most about 99%, 95%, 90%, 85%, 80%, 75%, 70%, 65%, 60%, 55%, 50%, 45%, 40%, 35%, 30%, 25%, 20%, 15%, 10%, 5%, 1%, of the nucleic acid construct(s), a vector(s), engineered polyribonucleotide(s), or compositions includes non-naturally occurring uracil, adenine, guanine, or cytosine.

The polynucleotides may comprise an open reading frame (ORF) sequence. The ORF sequence may be characterized by a codon usage profile comprising: (1) a total number of codons, (2) a species number of codons (e.g., a total number of different codon types), (3) a number of each (unique) codon, and (4) a (usage) frequency of each codon among all synonymous codons (if present). The codon usage profile may be altered or compared to a corresponding wild type sequence. For example, the frequency or number of particular codons may be reduced or increased compared to a wild type sequence. The change in codon frequency of the polynucleotide may provide benefits over the wild type sequence. For example, the altered codon frequency may result in a less immunogenic polynucleotide. The polynucleotide with an altered codon frequency may result in a polynucleotide that is more quickly expressed or results in a greater amount of expression product. The polynucleotide with an altered codon frequency may have increase stability, such as increased half-life in sera, or may be less susceptible to hydrolysis or other reactions that may result in the degradation of the polynucleotide.

TABLE 1
Example ORF sequences

| | | SEQ ID |
|---|---|---|
| Construct | DNA sequence (from 5′ to 3′) | NO. |
| DNAI1 | ATGATCCCAGCAAGCGCCAAGGCACCACACAAGCAGCCCCACAAGCAGAGC | 14 |
| GeneScript | ATCTCCATCGGCAGGGGCACAAGGAAGAGGGACGAGGATAGCGGAACCGAA | |
| Codon | GTGGGAGAGGGAACAGACGAGTGGGCACAGTCCAAGGCAACCGTGCGCCCA | |
| | CCTGACCAGCTGGAGCTGACAGATGCCGAGCTGAAGGAGGAGTTCACCAGG | |
| | ATCCTGACAGCCAACAATCCACACGCCCCCCAGAACATCGTGCGCTACTCT | |
| | TTCAAGGAGGGCACATATAAGCCAATCGGCTTTGTGAACCAGCTGGCCGTG | |
| | CACTATACCCAAGTGGGCAATCTGATCCCCAAGGACTCCGATGAGGGCCGG | |
| | AGACAGCACTACAGGGACGAGCTGGTGGCAGGATCCCAGGAGTCTGTGAAA | |
| | GTGATCTCTGAGACCGGCAATCTGGAGGAGGACGAGGAGCCAAAGGAGCTG | |
| | GAGACCGAGCCAGGAAGCCAGACAGATGTGCCTGCAGCAGGAGCAGCAGAG | |
| | AAGGTGACCGAGGAGGAGCTGATGACACCTAAGCAGCCAAAGGAGCGGAAG | |
| | CTGACCAACCAGTTCAATTTTTCCGAGAGAGCCTCTCAGACATACAACAAT | |
| | CCAGTGCGGGACAGAGAGTGCCAGACCGAGCCACCCCCTAGAACCAACTTT | |
| | TCCGCCACAGCCAATCAGTGGGAGATCTACGATGCCTATGTGGAGGAGCTG | |
| | GAGAAGCAGGAGAAGACCAAGGAGAAGGAGAAGGCCAAGACACCCGTGGCC | |
| | AAGAAGTCCGGCAAGATGGCCATGCGGAAGCTGACCAGCATGGAGTCCCAG | |
| | ACAGACGATCTGATCAAGCTGTCTCAGGCCGCCAAGATCATGGAGAGAATG | |
| | GTGAACCAGAATACCTATGACGATATCGCCCAGGACTTCAAGTACTATGAC | |
| | GATGCAGCAGACGAGTACAGGGATCAAGTGGGCACACTGCTGCCTCTGTGG | |
| | AAGTTTCAGAACGATAAGGCCAAGAGGCTGAGCGTGACCGCCCTGTGCTGG | |
| | AATCCAAAGTACAGGGACCTGTTCGCAGTGGGATACGGATCTTATGACTTC | |
| | ATGAAGCAGAGCAGAGGCATGCTGCTGCTGTATTCCCTGAAGAACCCCTCT | |
| | TTCCCTGAGTACATGTTTAGCTCCAATTCCGGCGTGATGTGCCTGGACATC | |
| | CACGTGGATCACCCCTACCTGGTGGCCGTGGGCCACTATGACGGCAACGTG | |
| | GCCATCTACAATCTGAAGAAGCCTCACTCTCAGCCCAGCTTCTGTTCTAGC | |
| | GCCAAGAGCGGCAAGCACTCCGATCCCGTGTGGCAGGTGAAGTGGCAGAAG | |
| | GACGATATGGACCAGAACCTGAATTTCTTTTCCGTGTCCTCTGATGGCAGG | |
| | ATCGTGTCTTGGACCCTGGTGAAGCGCAAGCTGGTGCACATCGACGTGATC | |
| | AAGCTGAAGGTGGAGGGCAGCACCACAGAGGTGCCAGAGGGACTGCAGCTG | |
| | CACCCAGTGGGATGCGGCACAGCCTTCGACTTTCACAAGGAGATCGATTAT | |
| | ATGTTCCTGGTGGGCACCGAGGAGGGCAAGATCTACAAGTGTTCTAAGAGC | |
| | TATAGCTCCCAGTTTCTGGACACATATGATGCCCACAACATGAGCGTGGAT | |
| | ACCGTGTCCTGGAATCCTTACCACACAAAGGTGTTCATGAGCTGCTCTAGC | |
| | GACTGGACCGTGAAGATCTGGGATCACACCATCAAGACACCTATGTTTATC | |
| | TATGACCTGAACTCCGCCGTGGGCGATGTGGCATGGGCACCATACTCCTCT | |
| | ACAGTGTTCGCAGCAGTGACCACAGACGGCAAGGCACACATCTTTGATCTG | |
| | GCCATCAACAAGTACGAGGCCATCTGTAATCAGCCCGTGGCCGCCAAGAAG | |
| | AACAGGCTGACCCACGTGCAGTTCAATCTGATCCACCCTATCATCATCGTG | |
| | GGCGACGATCGGGGCCACATCATCTCTCTGAAGCTGAGCCCCAACCTGAGA | |
| | AAGATGCCTAAGGAGAAGAAGGGACAGGAGGTGCAGAAGGGACCAGCAGTG | |
| | GAGATCGCAAAGCTGGACAAGCTGCTGAATCTGGTGCGCGAGGTGAAGATC | |
| | AAGACCTGA | |
| DNAI1 | ATGATCCCAGCAAGCGCCAAGGCACCACACAAGCAGCCCCACAAGCAGAGC | 15 |
| Altered | ATCAGCATCGGCAGGGGCACAAGGAAGAGGGACGAGGACAGCGGAACCGAA | |
| Nucleotide | GTGGGAGAGGGAACAGACGAGTGGGCACAGAGCAAGGCAACCGTGCGCCCA | |
| Usage 1 | CCCGACCAGCTGGAGCTGACAGACGCCGAGCTGAAGGAGGAGTTCACCAGG | |
| | ATCCTGACAGCCAACAACCCACACGCCCCCCAGAACATCGTGCGCTACAGC | |
| | TTCAAGGAGGGCACATACAAGCCAATCGGCTTCGTGAACCAGCTGGCCGTG | |
| | CACTACACCCAAGTGGGCAACCTGATCCCCAAGGACAGCGACGAGGGCCGG | |
| | AGACAGCACTACAGGGACGAGCTGGTGGCAGGAAGCCAGGAGAGCGTGAAA | |
| | GTGATCAGCGAGACCGGCAACCTGGAGGAGGACGAGGAGCCAAAGGAGCTG | |
| | GAGACCGAGCCAGGAAGCCAGACAGACGTGCCCGCAGCAGGAGCAGCAGAG | |
| | AAGGTGACCGAGGAGGAGCTGATGACACCCAAGCAGCCAAAGGAGCGGAAG | |
| | CTGACCAACCAGTTCAACTTCAGCGAGAGAGCCAGCCAGACATACAACAAC | |
| | CCAGTGCGGGACAGAGAGTGCCAGACCGAGCCACCCCCCAGAACCAACTTC | |
| | AGCGCCACAGCCAACCAGTGGGAGATCTACGACGCCTACGTGGAGGAGCTG | |
| | GAGAAGCAGGAGAAGACCAAGGAGAAGGAGAAGGCCAAGACACCCGTGGCC | |
| | AAGAAGAGCGGCAAGATGGCCATGCGGAAGCTGACCAGCATGGAGAGCCAG | |
| | ACAGACGACCTGATCAAGCTGAGCCAGGCCGCCAAGATCATGGAGAGAATG | |
| | GTGAACCAGAACACCTACGACGACATCGCCCAGGACTTCAAGTACTACGAC | |
| | GACGCAGCAGACGAGTACAGGGACCAAGTGGGCACACTGCTGCCCCTGTGG | |
| | AAGTTCCAGAACGACAAGGCCAAGAGGCTGAGCGTGACCGCCCTGTGCTGG | |
| | AACCCAAAGTACAGGGACCTGTTCGCAGTGGGATACGGAAGCTACGACTTC | |
| | ATGAAGCAGAGCAGAGGCATGCTGCTGCTGTACAGCCTGAAGAACCCCAGC | |
| | TTCCCCGAGTACATGTTCAGCAGCAACAGCGGCGTGATGTGCCTGGACATC | |
| | CACGTGGACCACCCCTACCTGGTGGCCGTGGGCCACTACGACGGCAACGTG | |
| | GCCATCTACAACCTGAAGAAGCCCCACAGCCAGCCCAGCTTCTGCAGCAGC | |
| | GCCAAGAGCGGCAAGCACAGCGACCCCGTGTGGCAGGTGAAGTGGCAGAAG | |
| | GACGACATGGACCAGAACCTGAACTTCTTCAGCGTGAGCAGCGACGGCAGG | |
| | ATCGTGAGCTGGACCCTGGTGAAGCGCAAGCTGGTGCACATCGACGTGATC | |
| | AAGCTGAAGGTGGAGGGCAGCACCACAGAGGTGCCAGAGGGACTGCAGCTG | |
| | CACCCAGTGGGATGCGGCACAGCCTTCGACTTCCACAAGGAGATCGACTAC | |
| | ATGTTCCTGGTGGGCACCGAGGAGGGCAAGATCTACAAGTGCAGCAAGAGC | |
| | TACAGCAGCCAGTTCCTGGACACATACGACGCCCACAACATGAGCGTGGAC | |
| | ACCGTGAGCTGGAACCCCTACCACACAAAGGTGTTCATGAGCTGCAGCAGC | |
| | GACTGGACCGTGAAGATCTGGGACCACACCATCAAGACACCCATGTTCATC | |
| | TACGACCTGAACAGCGCCGTGGGCGACGTGGCATGGGCACCATACAGCAGC | |
| | ACAGTGTTCGCAGCAGTGACCACAGACGGCAAGGCACACATCTTCGACCTG | |
| | GCCATCAACAAGTACGAGGCCATCTGCAACCAGCCCGTGGCCGCCAAGAAG | |
| | AACAGGCTGACCCACGTGCAGTTCAACCTGATCCACCCCATCATCATCGTG | |
| | GGCGACGACCGGGGCCACATCATCAGCCTGAAGCTGAGCCCCAACCTGAGA | |
| | AAGATGCCCAAGGAGAAGAAGGGACAGGAGGTGCAGAAGGGACCAGCAGTG | |
| | GAGATCGCAAAGCTGGACAAGCTGCTGAACCTGGTGCGCGAGGTGAAGATC | |
| | AAGACCTGA | |
| Wild Type | ATGATTCCTGCTTCTGCGAAGGCTCCCCATAAACAGCCTCATAAGCAGAGC | 16 |
| DNAI1 | ATCAGCATAGGCAGAGGAACCAGGAAGAGAGATGAAGATTCAGGGACTGAA | |
| | GTGGGAGAAGGCACAGATGAATGGGCCCAATCCAAAGCCACAGTTAGACCC | |
| | CCTGACCAGCTGGAGTTGACCGATGCGGAGTTAAAGGAGGAGTTCACTCGG | |
| | ATTTTGACAGCCAACAACCCACACGCACCCCAGAACATTGTCAGGTACAGC | |
| | TTCAAAGAAGGCACATATAAGCCTATTGGCTTTGTGAACCAACTGGCAGTT | |
| | CACTACACCCAGGTTGGGAACCTGATCCCCAAAGACTCAGATGAAGGACGG | |
| | CGGCAGCATTACCGCGATGAATTAGTGGCAGGTTCTCAGGAGTCTGTCAAG | |
| | GTGATTTCAGAAACAGGAAACCTCGAAGAAGACGAAGAGCCCAAGGAGTTA | |
| | GAAACTGAGCCTGGGAGTCAAACAGATGTGCCTGCAGCTGGGGCAGCTGAA | |
| | AAAGTGACTGAAGAAGAATTGATGACTCCTAAGCAGCCCAAGGAGAGAAAG | |
| | CTCACTAACCAGTTCAACTTCAGTGAGAGGGCCTCACAGACCTACAACAAC | |
| | CCTGTCCGGGATCGAGAATGCCAGACGGAGCCTCCTCCCAGGACAAACTTT | |
| | TCAGCCACAGCCAATCAGTGGGAGATCTATGATGCCTATGTAGAGGAACTT | |
| | GAGAAGCAGGAAAAGACCAAAGAGAAGGAGAAGGCAAAGACCCCAGTGGCT | |
| | AAAAAATCAGGGAAGATGGCCATGAGGAAGCTGACATCTATGGAGTCTCAG | |
| | ACTGATGATCTCATCAAATTGTCCCAAGCTGCTAAGATCATGGAGCGGATG | |
| | GTCAACCAGAATACATATGATGACATTGCTCAAGATTTTAAGTACTATGAC | |
| | GATGCTGCTGATGAATACCGGGACCAGGTGGGTACCCTGCTGCCGCTCTGG | |
| | AAGTTCCAAAATGACAAAGCCAAGCGCCTGTCCGTCACTGCCCTCTGCTGG | |
| | AATCCAAAGTACAGGGATCTGTTTGCAGTGGGATATGGCTCTTATGACTTC | |
| | ATGAAGCAGAGCCGGGGCATGCTGCTGCTCTACAGCCTGAAGAACCCCAGC | |
| | TTCCCTGAGTACATGTTCAGCAGCAACAGCGGCGTCATGTGTCTCGACATC | |
| | CACGTGGACCACCCCTACCTGGTGGCAGTAGGCCACTATGACGGCAACGTG | |
| | GCCATTTACAACCTCAAGAAGCCCCACTCCCAGCCCTCCTTCTGCAGCTCA | |
| | GCCAAGTCTGGCAAGCACTCAGACCCTGTGTGGCAGGTCAAGTGGCAGAAG | |
| | GATGACATGGACCAAAACCTTAACTTCTTCTCTGTGTCATCTGACGGCAGG | |
| | ATTGTGTCTTGGACTCTCGTGAAGAGAAAGCTGGTTCACATAGATGTCATC | |
| | AAGCTGAAGGTGGAAGGCAGCACCACGGAAGTTCCTGAGGGGTTGCAGCTG | |
| | CACCCAGTGGGTTGTGGCACTGCCTTTGACTTCCACAAAGAGATTGACTAC | |
| | ATGTTCCTAGTGGGCACAGAGGAGGGAAAAATCTACAAGTGCTCTAAATCC | |
| | TACTCCAGCCAATTCCTCGACACCTATGACGCCCACAACATGTCAGTGGAC | |
| | ACTGTGTCCTGGAACCCATACCACACCAAGGTCTTCATGTCCTGCAGCTCC | |
| | GACTGGACAGTGAAGATCTGGGACCACACCATCAAGACCCCGATGTTCATC | |
| | TATGACCTGAACTCAGCCGTGGGTGATGTGGCCTGGGCGCCATACTCTTCT | |
| | ACTGTGTTCGCAGCAGTCACCACAGATGGGAAGGCCCACATATTTGACTTA | |
| | GCCATCAACAAGTATGAGGCCATCTGCAACCAGCCTGTGGCGGCCAAAAAG | |
| | AACAGGCTCACCCACGTGCAGTTCAATCTCATCCACCCCATCATCATTGTG | |
| | GGCGATGACCGTGGGCACATCATCAGCCTCAAGCTCTCACCCAATTTGCGC | |
| | AAGATGCCAAAGGAAAAGAAGGGGCAGGAGGTGCAGAAGGGTCCAGCTGTG | |
| | GAGATTGCGAAACTGGACAAACTGCTGAACCTGGTGAGGGAAGTGAAAATC | |
| | AAGACCTGA | |

### Codon Optimizations

In some embodiments, the polynucleotide comprises an altered nucleotide usage as compared to a corresponding wild type sequence. The altered nucleotide usage may also be referred to as a “codon optimized” sequence or be generated by way of “codon optimization”. The codon optimized polynucleotides may comprise

Altered nucleotide usage schemes aiming to reduce the number of more reactive 5′-U(U/A)-3′ dinucleotides within codons as well as across codons of modified mRNAs partially alleviate limitations imposed by the inherent chemical instability of RNA. At the same time, lowering the U-content in RNA transcripts renders them less immunogenic. The present disclosure relates to RNA transcripts comprising altered open reading frames (ORF). For example, the codon optimized or altered nucleotide usage may comprise a substantial reduction of 5′-U(U/A)-3′ dinucleotides within protein coding regions leading to stabilized therapeutic mRNAs. The codon optimized polynucleotide may comprise a codon coding for a particular amino acid to be substituted or replaced of a with a synonymous codon. The codon optimized polynucleotide may encode a same or identical polypeptide as a corresponding wild type polynucleotide, with the polynucleotide comprising a different sequence of polynucleotide than the corresponding wild type. Multiple codons may encode for a same amino acid, however the qualities of a given codon are differ between even those that code for a same amino acid. Because multiple different codons may code for a same amino acid, a particular polynucleotide may encode for a same polypeptide and have advantageous features over another polynucleotide that codes for the same polypeptide. For example, a codon optimized polynucleotides may be transcribed faster, may comprise a higher stability (in vivo or in vitro), may result in increased expression yield or full length or functional polypeptides, or may result in an increase of soluble polypeptide and a decrease in polypeptide aggregates. Without being limited to a specific mechanism, the advantageous features of a codon optimized polynucleotides may be for example, a result of improved protein folding of the expressed product based on ribosomal interactions with the polynucleotides, or may be result of decreased hydrolysis of reactive bonds in solution. For example, the codon optimization may be alter or improve characteristics relating to ribosomal binding sites, Shine-Dalgarno sequences, or ribosomal or translational pausing. The advantageous features may be a result of decreased usage of “rare codons” which may have a lower concentration of cognate tRNAs, allowing for an improved translation reaction. The advantageous features may be a result of decreased usage of “rare codons” which may have a lower concentration of cognate tRNAs, allowing for an improved translation reaction. The advantageous features may be a result of decreasing degradation via enzymatic reaction. For example, hydrolysis of oligonucleotides suggests that the reactivity of the phosphodiester bond linking two ribonucleotides in single-stranded (ss)RNA depends on the nature of those nucleotides. At pH 8.5, dinucleotide cleavage susceptibility when embedded in ssRNA dodecamers may vary by an order of magnitude. Under near physiological conditions, hydrolysis of RNA usually involves an S_{N}2-type attack by the 2′-oxygen nucleophile on the adjacent phosphorus target center on the opposing side of the 5′-oxyanion leaving group, yielding two RNA fragments with 2′,3′-cyclic phosphate and 5′-hydroxyl termini. More reactive scissile phosphodiester bonds may include 5′-UpA-3′ (R₁═U₁, R₂=A) and 5′-CpA-3′ (R₁═C, R₂=A) because the backbone at these steps can most easily adopt the “in-line” conformation that is required for S_{N}2-type nucleophilic attack by the 2′-OH on the adjacent phosphodiester linkage. In addition, interferon-regulated dsRNA-activated antiviral pathways produce 2′-5′ oligoadenylates which bind to ankyrin repeats leading to activation of RNase L endoribonuclease. RNase L cleaves ssRNA efficiently at UA and UU dinucleotides. Lastly, U-rich sequences are potent activators of RNA sensors including Toll-like receptor 7 and 8 and RIG-I making global uridine content reduction a potentially attractive approach to reduce immunogenicity of therapeutic mRNAs.

In some embodiments, the nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CAG, AGG, CGG, CGA, CGT, CGC, TCG, TCA, TCT, TCC, ACG, ACT, GTA, GTT, GTC, and TAT, as compared to a corresponding wild-type sequence, e.g., SEQ ID NO: 16. In some embodiments, the nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence, e.g., SEQ ID NO: 16. In some embodiments, the nucleic acid sequence comprises fewer codon types encoding an amino acid as compared to a corresponding wild-type sequence, e.g., SEQ ID NO: 16.

In some cases, a codon coding for a particular amino acid in the polypeptide may be substituted or replaced with a synonymous codon. For example, a codon coding for leucine may be substituted for another codon coding for leucine. In this way, the resulting translation products may be identical with the polynucleotide differing in sequence. At least one type of an isoleucine-encoding codons in the corresponding wild-type sequence may be substituted with a synonymous codon type in the nucleic acid sequence. At least one type of a valine-encoding codons in the corresponding wild-type sequence may be substituted with a synonymous codon type in the nucleic acid sequence. At least one type of an alanine-encoding codons in the corresponding wild-type sequence may be substituted with a synonymous codon type in the nucleic acid sequence. At least one type of a glycine-encoding codons in the corresponding wild-type sequence may be substituted with a synonymous codon type in the nucleic acid sequence. At least one type of a proline-encoding codons in the corresponding wild-type sequence may be substituted with a synonymous codon type in the nucleic acid sequence. At least one type of a threonine-encoding codons in the corresponding wild-type sequence may be substituted with a synonymous codon type in the nucleic acid sequence. At least one type of a leucine-encoding codons in the corresponding wild-type sequence may be substituted with a synonymous codon type in the nucleic acid sequence. At least one type of an arginine-encoding codons in the corresponding wild-type sequence is substituted with a synonymous codon type in the nucleic acid sequence. At least one type of a serine-encoding codons in the corresponding wild-type sequence may be substituted with a synonymous codon type in the nucleic acid sequence.

In some aspects described herein, a particular codon of a particular amino acid comprises a percentage or amount of the total number of codons for that particular amino acid the polynucleotide. This may be referred to a “codon frequency”. For example, at least 50% of the total codons encoding a particular amino acid in the polynucleotide may be encoded by a first codon sequence. For example, at least 55% of the total codons encoding a particular amino acid in the polynucleotide may be encoded by a first codon sequence. At least 5%, 10%, 20%, 30%, 40%, 50%, 55%, 60%, 65%, 70%, 75%, 80%, 85%, 90%, 95%, or more of the total codons encoding a particular amino in the polynucleotide may be encoded by a first codon sequence. In some cases, no more than 5%, 10%, 20%, 30%, 40%, 50%, 55%, 60%, 65%, 70%, 75%, 80%, 85%, 90%, 95%, or less of the total codons encoding a particular amino in the polynucleotide are encoded by a first codon sequence. At least about 90% phenylalanine-encoding codons of the synthetic polynucleotide may be TTC (as opposed to TTT). At least about 60% cysteine-encoding codons of the synthetic polynucleotide may be TGC (as opposed to TGT). At least about 70% aspartic acid-encoding codons of the synthetic polynucleotide may be GAC (as opposed to GAT). At least about 50% glutamic acid-encoding codons of the synthetic polynucleotide may be GAG (as opposed to GAA). At least about 60% histidine-encoding codons of the synthetic polynucleotide may be CAC (as opposed to CAT). At least about 60% lysine-encoding codons of the synthetic polynucleotide may be AAG (as opposed to AAA). At least about 60% asparagine-encoding codons of the synthetic polynucleotide may be AAC (as opposed to AAT). At least about 70% glutamine-encoding codons of the synthetic polynucleotide may be CAG (as opposed to CAA). At least about 80% tyrosine-encoding codons of the synthetic polynucleotide may be TAC (as opposed to TAT). At least about 90% isoleucine-encoding codons of the synthetic polynucleotide may be ATC.

In some embodiments, a particular amino acid the polynucleotide may be encoded by a number of different codon sequences. For example, a particular amino acid in the polynucleotide may be encoded by no more than 2 different codon sequences. In some cases, the polynucleotide comprises no more than 2 types of isoleucine-encoding codons.

In some embodiments, a particular amino acid in the polynucleotide may be encoded by no more than 3 different codon sequences. The polynucleotide may comprise no more than 3 types of alanine (Ala)-encoding codons. The polynucleotide may comprise no more than 3 types of glycine (Gly)-encoding codons. The polynucleotide may comprise no more than 3 types of proline (Pro)-encoding codons. The polynucleotide may comprise no more than 3 types of threonine (Thr)-encoding codons.

In some embodiments, a particular amino acid in the polynucleotide may be encoded by no more than 4 different codon sequences. The polynucleotide may comprise no more than 4 types of arginine (Arg)-encoding codons. The polynucleotide may comprise no more than 4 types of serine (Ser)-encoding codons. In some embodiments, a particular amino acid in the polynucleotide may be encoded by no more than 5 different codon sequences. The polynucleotide may comprise no more than 5 types of arginine (Arg)-encoding codons. The polynucleotide may comprise no more than 5 types of serine (Ser)-encoding codons. In some embodiments, a particular amino acid in the polynucleotide may be encoded by no more than 6 different codon sequences. In some embodiments, a particular amino acid in the polynucleotide may be encoded by 1 or more different codon sequences. In some embodiments, a particular amino acid in the polynucleotide may be encoded by 2 or more different codon sequences. In some embodiments, a particular amino acid in the polynucleotide may be encoded by 3 or more different codon sequences. In some embodiments, a particular amino acid in the polynucleotide may be encoded by 4 or more different codon sequences. In some embodiments, a particular amino acid in the polynucleotide may be encoded by 5 or more different codon sequences. In some embodiments, a particular amino acid in the polynucleotide may be encoded by 6 or more different codon sequences.

In some cases, a frequency of a first codon sequence of a is higher, lower or the same as a frequency of a second codon sequence encoding for a particular amino acid in the polynucleotide. For example, a frequency of a first codon is higher than a frequency of second codon for a particular amino acid in the polynucleotide. The frequency of GCC codon may be higher than a frequency of GCT codon. The frequency of GCT codon may be lower than a frequency of GCA codon. The frequency of GCT codon may be higher than a frequency of GCA codon.

In some embodiments, the codon usage for alanine-encoding codons in the polynucleotide may have a particular parameter. For example, a frequency of GCG codon may be no more than about 10% or 5%.

A frequency of GCA codon may be no more than about 20%. A frequency of GCT codon may be at least about 1%, 5%, 10%, 15%, 20%, or 25%. A frequency of GCT codon may be no more than about 30%, 25%, 20%, 15%, 10%, or 5%. A frequency of GCC codon may be at least about 60%, 70%, 80%, or 90%. A frequency of GCC codon is no more than about 95%, 90%, 85%, 80%, or 75%. The frequency of GCC codon may be higher than a frequency of GCT codon. The frequency of GCT codon may be lower than a frequency of GCA codon. The frequency of GCT codon may be higher than a frequency of GCA codon.

In some embodiments, the codon usage for glycine-encoding codons the polynucleotide may have a particular parameter. For example, a frequency of GGC codon may be lower than a frequency of GGA codon. For example, a frequency of GGC codon may be higher than a frequency of GGA codon. A frequency of GGG codon may be no more than about 10% or 5%. A frequency of GGG codon may be least about 1%. A frequency of GGA codon may be no more than about 30% or 20%. A frequency of GGA codon may be at least about 10% or 20%. A frequency of GGT codon may be more than about 10% or 5%. A frequency of GGC codon may be no more than about 90%, 80%, or 70%. A frequency of GGC codon may be least about 60%, 70%, or 80%.

In some embodiments, the codon usage for proline-encoding codons the polynucleotide may have a particular parameter. For example, a frequency of CCC codon may be lower than a frequency of CCT codon. A frequency of CCC codon may be higher than a frequency of CCT codon. A frequency of CCC codon may be lower than a frequency of CCA codon. A frequency of CCC codon may be higher than a frequency of CCA codon. A frequency of CCT codon may be lower than a frequency of CCA codon. A frequency of CCT codon may be higher than a frequency of CCA codon. A frequency of CCG codon may be no more than about 10% or 5%. frequency of CCA codon may be no more than about 30%, 20%, or 10%. A frequency of CCA codon may be at least about 5%, 10%, 15%, 20%, or 25%. A frequency of CCT codon may be no more than about 60%, 50%, 40%, or 30%. A frequency of CCT codon may be at least about 20%, 30%, 40%, or 50%. A frequency of CCC codon may be no more than about 60%, 50%, or 40%. A frequency of CCC codon may be at least about 30%, 40%, 50%, 60%, or 70%.

In some embodiments, the codon usage for threonine-encoding codons the polynucleotide may have a particular parameter. For example, a frequency of ACA codon is higher than a frequency of ACT codon. A frequency of ACC codon may be higher than a frequency of ACT codon. A frequency of ACC codon may be lower than a frequency of ACA codon. A frequency of ACC codon may be higher than a frequency of ACA codon. A frequency of ACG codon may be no more than about 10% or 5%. A frequency of ACA codon may be no more than about 60%, 50%, 40%, or 30%. A frequency of ACA codon may be at least about 10%, 20%, 30%, 40%, or 50%. A frequency of ACT codon may be no more than about 10% or 5%. A frequency of ACC codon may be no more than about 90%, 80%, 70%, 60%, or 50%. A frequency of ACC codon is at least about 40%, 50%, 60%, 70%, or 80%.

In some embodiments, the codon usage for arginine-encoding codons the polynucleotide may have a particular parameter. For example, a frequency of AGA codon may be lower than a frequency of AGG codon. A frequency of AGA codon may be higher than a frequency of AGG codon. A frequency of AGA codon may be lower than a frequency of CGG codon. A frequency of AGA codon may be higher than a frequency of CGG codon. A frequency of CGG codon may be higher than a frequency of CGA codon. A frequency of CGG codon is higher than a frequency of CGC codon. A frequency of AGG codon may be no more than about 10%. A frequency of AGG codon may be less than about 10%. A frequency of AGA codon may be no more than about 70%, 60%, or 50%. A frequency of AGA codon may be at least about 40%, 50%, 60%, or 70%. A frequency of CGG codon may be no more than about 50%, 40%, or 30%. A frequency of CGG codon may be at least about 20%, 30%, or 40%. A frequency of CGA codon may be at least about 1%. A frequency of CGA codon may be no more than about 10% or 5%. A frequency of CGT codon may be no more about 10% or 5%. A frequency of CGC codon may be no more than about 20%, 10%, or 5%. A frequency of CGC codon may be at least about 1%, 2%, 3%, 4%, or 5%.

In some embodiments, the codon usage for serine-encoding codons the polynucleotide may have a particular parameter. For example, a frequency of AGC codon may be higher than a frequency of TCT codon. A frequency of TCT codon may be higher than a frequency of TCG codon. A frequency of TCT codon may be higher than a frequency of TCA codon. A frequency of TCT codon may be higher than a frequency of TCC codon. A frequency of AGT codon may be no more than about 10%. A frequency of AGT codon may be at least about 1%. A frequency of AGC codon may be no more about 95%, 90%, 85%, or 80%. A frequency of AGC codon may be at least about 70%, 80%, or 90%. A frequency of TCG codon may be no more than about 10% or 5%. A frequency of TCA codon may be no more than about 10% or 5%. A frequency of TCT codon may be no more than about 30%, 20%, or 10%. A frequency of TCT codon may be at least about 10%, or 20%. A frequency of TCC codon may be no more than about 10% or 5%.

### Untranslated Regions

In some instances, a polynucleotide, nucleic acid construct, vector, or composition of the disclosure comprises one or more nucleotide sequences that encode dynein axonemal intermediate chain 1 (DNAI1) protein or a variant thereof, and the sequences provide for heterologous or enhanced expression of the dynein axonemal intermediate chain 1 (DNAI1) protein or a variant thereof within cells of a subject. In some instances, the nucleic acid construct, vector, or composition also comprises a 5′ untranslated region (UTR) or 3′ UTR having at least 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to one set forth in SEQ ID NOs 1-14, SEQ ID NOs. 1-8 and 14, or SEQ ID NOs. 8-13. In some instances, the polynucleotide comprises a 5′ untranslated region (UTR) having at least 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID 14. In some embodiments, the nucleic acid sequence of the present disclosure comprises one or more (e.g., one or two) sequences set forth in SEQ ID NOs 1-8 and 14. In some embodiments, the nucleic acid sequence of the present disclosure comprises a sequence set forth in SEQ ID NOs 8-13. In some embodiments, the nucleic acid sequence of the present disclosure comprises the sequence set forth in SEQ ID NO 14.

TABLE 2
Example UTR sequences

| | | SEQ ID |
|---|---|---|
| UTR | DNA sequence (from 5′ to 3′) | NO. |
| α-globin 5′ | GGGAGACATAAACCCTGGCGCGCTCGCGGCCCGGCACTCTTCTGGT | 1 |
| UTR (HBA1) | CCCCACAGACTCAGAGAGAAGCCACC | |
| α-globin 5′ | GGGAGACATAAACCCTGGCGCGCTCGCGGGCCGGCACTCTTCTGGT | 2 |
| UTR (HBA2) | CCCCACAGACTCAGAGAGAAGCCACC | |
| α-globin 5′ | GGGAGACTCTTCTGGTCCCCACAGACTCAGAGAGAACGCCACC | 3 |
| UTR | | |
| IRES of | GTTATTTTCCACCATATTGCCGTCTTTTGGCAATGTGAGGGCCCGG | 4 |
| EMCV 5′- | AAACCTGGCCCTGTCTTCTTGACGAGCATTCCTAGGGGTCTTTCCC | |
| UTR | CTCTCGCCAAAGGAATGCAAGGTCTGTTGAATGTCGTGAAGGAAGC | |
| | AGTTCCTCTGGAAGCTTCTTGAAGACAAACAACGTCTGTAGCGACC | |
| | CTTTGCAGGCAGCGGAACCCCCCACCTGGCGACAGGTGCCTCTGCG | |
| | GCCAAAAGCCACGTGTATAAGATACACCTGCAAAGGCGGCACAACC | |
| | CCAGTGCCACGTTGTGAGTTGGATAGTTGTGGAAAGAGTCAAATGG | |
| | CTCTCCTCAAGCGTATTCAACAAGGGGCTGAAGGATGCCCAGAAGG | |
| | TACCCCATTGTATGGGATCTGATCTGGGGCCTCGGTGCACATGCTT | |
| | TACGTGTGTTTAGTCGAGGTTAAAAAACGTCTAGGCCCCCCGAACC | |
| | ACGGGGACGTGGTTTTCCTTTGAAAAACACGATGATAATATGGCCA | |
| | CAACC | |
| IRES of TEV | AAATAACAAATCTCAACACAACATATACAAAACAAACGAATCTCAA | 5 |
| 5′-UTR | GCAATCAAGCATTCTACTTCTATTGCAGCAATTTAAATCATTTCTT | |
| | TTAAAGCAAAAGCAATTTTCTGAAAATTTTCACCATTTACGAACGA | |
| | TAGCA | |
| SSRNA1 | GGGAGACAAGAGAGAAAAGAAGAGCAAGAAGAAATATAAGAGCCAC | 6 |
| 5′UTR | C | |
| SSRNA2 | GGGAGACCCAAGCTGGCTAGCGTTTAAACTTAAGCTTGGCAATCCG | 7 |
| 5′UTR | GTACTGTTGGTAAAGCCACC | |
| SSRNA 3 + | GGGAGACCCAAGCTGGCTAGCGTTTAAACTTAAGCTTTCCTTTCCG | 8 |
| native 5′ | GGCCGGCTGGGCGCGCCGAAGCGCCTGCGCCTTGGCTGCTGGTCGG | |
| UTR | TTGCTGGGTAACCGCGTCAGGGAGTTGGATTCTATCCTGCAAGGGC | |
| | ACGGGGACCCACAACGACGGCTGTCCCTAAAGAACCGTTGCGACTG | |
| | GTAACTGAAGTGGAAGAGAGTCCAGATTTCTTGTGTGTGGTCAAGG | |
| | AGACGGACAAACTTTTTGTCTTCAGACGAGGGAGCGTTTTGTAGGC | |
| | TCTCCAGGGGTTGAG | |
| TMV 3′-UTR | GGATTGTGTCCGTAATCACACGTGGTGCGTACGATAACGCATAGTG | 9 |
| | TTTTTCCCTCCACTTAAATCGAAGGGTTGTGTCTTGGATCGCGCGG | |
| | GTCAAATGTATATGGTTCATATACATCCGCAGGCACGTAATAAAGC | |
| | GAGGGGTTCGAATCCCCCCGTTACCCCCGGTAGGGGCCCATTGTCT | |
| | TC | |
| MALAT1 3′- | TCAGTAGGGTCATGAAGGTTTTTCTTTTCCTGAGAAAACAACACGT | 10 |
| UTR | ATTGTTTTCTCAGGTTTTGCTTTTTGGCCTTTTTCTAGCTTAAAAA | |
| | AAAAAAAAGCAAAATTGTCTTC | |
| NEAT2 3′- | TCAGTAGGGTTGTAAAGGTTTTTCTTTTCCTGAGAAAACAACCTTT | 11 |
| UTR | TGTTTTCTCAGGTTTTGCTTTTTGGCCTTTCCCTAGCTTTAAAAAA | |
| | AAAAAAGCAAAATTGTCTTC | |
| histone | GAAGTGGCGGTTCGGCCGGAGGTTCCATCGTATCCAAAAGGCTCTT | 12 |
| cluster 2, | TTCAGAGCCACCCATTGTCTTC | |
| H3c 3′-UTR | | |
| Native 3′ | GGGGCTGGCCTCAGTCTCTGTCCCATCGCTTGAATACAGTACTCCT | 13 |
| UTR | AGGGCTTGACCCTGGTACCCAGCCCAGCCTTAGCACCCAGCATGTG | |
| | ACCCCACTCCTGATCAGGTCCCAGCATCTTCCCTTCTTGTTCTGTT | |
| | CCTTAAGGTCCCAGCACCTTACCCCAGGACTTGGTCTTCAACCACC | |
| | ATTACCCCTCTAACTTTGCACAAATAAACCTGTGTAGAAACCCACC | |
| | CCAAAAAAA | |
| ssRNA2 | GGGAGACCCAAGCTGGCTAGCGTTTAAACTTCAGCTTGGCAATCCG | 14 |
| 5′UTR | GTACTGTTGGTAAAGCCACC | |
| (A32C) | | |

In some embodiments, the polynucleotide is present in the (e.g., pharmaceutical) composition at a concentration of no more than 1 mg/mL. In some embodiments, the polynucleotide is present in the (e.g., pharmaceutical) composition at a concentration of no more than 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or less. In some embodiments, the polynucleotide is present in the (e.g., pharmaceutical) composition at a concentration of at least 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or more. In some embodiments, the polynucleotide is present in the (e.g., pharmaceutical) composition at a concentration of any one of the following values or within a range of any two of the following values: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or a range between any two of the foregoing values. In some embodiments, the polynucleotide is present in the (e.g., pharmaceutical) composition at a concentration from 0.5 mg/mL to 5 mg/mL. In some embodiments, the polynucleotide is present in the (e.g., pharmaceutical) composition at a concentration from 0.5 mg/mL to 1 mg/mL. In some embodiments, the polynucleotide is present in the (e.g., pharmaceutical) composition at a concentration from 2 mg/mL to 5 mg/mL.

### Lipid Formulations

The present disclosure provides a (e.g., pharmaceutical) composition comprising a polynucleotide assembled with a lipid composition, wherein the polynucleotide encodes a dynein axonemal intermediate chain 1 (DNAI1) protein; and wherein the lipid composition comprises a (e.g., ionizable) cationic lipid. The polynucleotide may be a polynucleotide as disclosed hereinabove or disclosed elsewhere herein. The polynucleotide may comprise a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15.

### LF92 Formulations

In an aspect, the lipid composition of the present disclosure comprises a cationic lipid having a structural formula (I′):

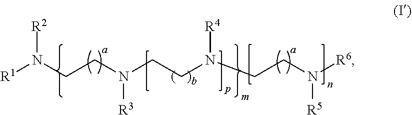

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I′) may be protonated to provide a cationic lipid.

In some embodiments of the cationic lipid of formula (I′), a is 1. In some embodiments of the cationic lipid of formula (I′), b is 2. In some embodiments of the cationic lipid of formula (I′), m is 1. In some embodiments of the cationic lipid of formula (I′), n is 1. In some embodiments of the cationic lipid of formula (I′), R¹, R², R³, R⁴, R⁵, and R⁶ are each independently H or —CH₂CH(OH)R⁷. In some embodiments of the cationic lipid of formula (I′), R¹, R², R³, R⁴, R⁵, and R⁶ are each independently H or

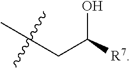

In some embodiments of the cationic lipid of formula (I′), R¹, R², R³, R⁴, R⁵, and R⁶ are each independently H or

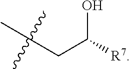

In some embodiments of the cationic lipid of formula (I′), R⁷ is C₃-C₁₈ alkyl (e.g., C₆-C₁₂ alkyl).

In some embodiments, the cationic lipid of formula (I′) is 13,16,20-tris(2-hydroxydodecyl)-13,16,20,23-tetraazapentatricontane-11,25-diol:

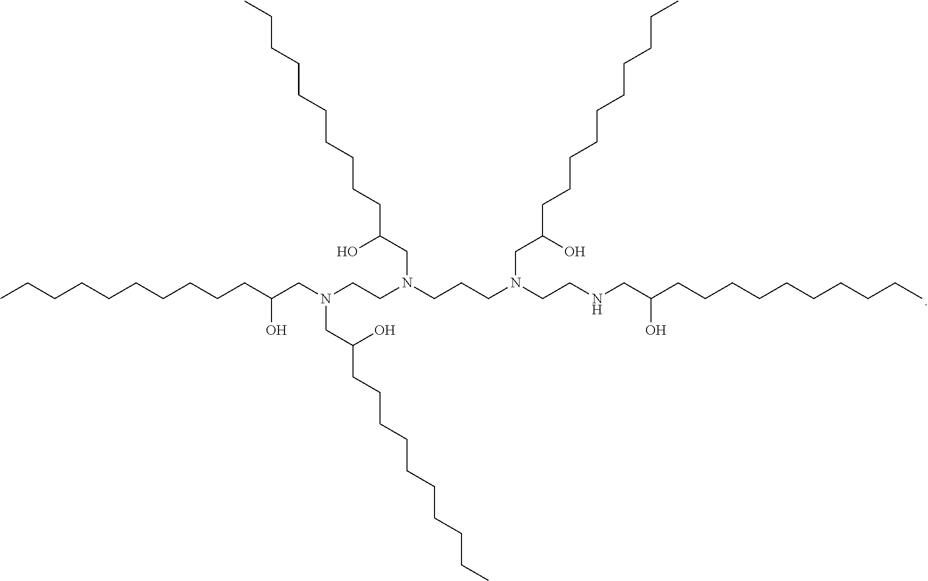 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (I′) is (11R,25R)-13,16,20-tris((R)-2-hydroxydodecyl)-13,16,20,23-tetraazapentatricontane-11,25-diol:

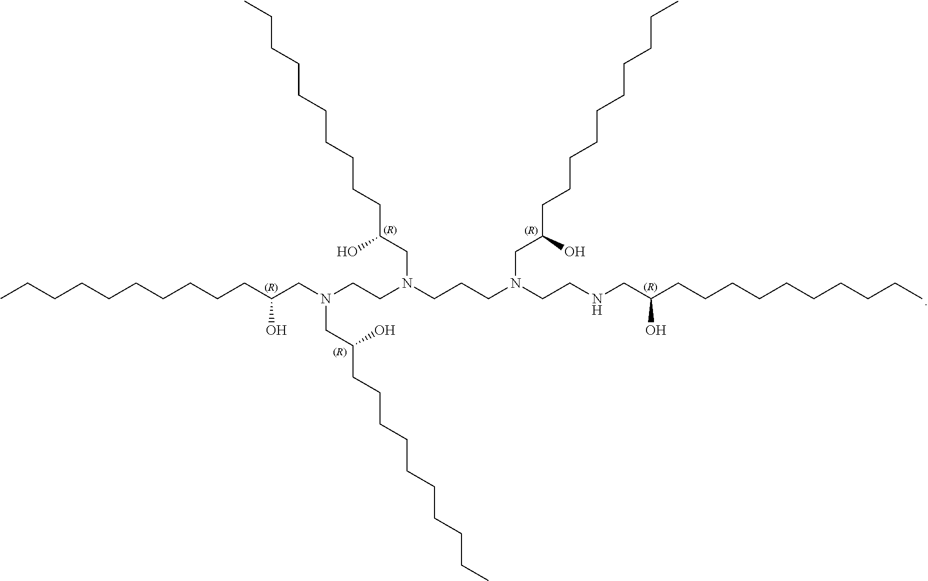

In some embodiments of the LF92 lipid composition, a lipid of the lipid composition can be in a particular amount or molar percentage. In some embodiments, the lipid composition comprises the cationic lipid of formula (I′) at a molar percentage of no more than 50% (e.g., no more than 45%). In some embodiments, the LF92 lipid composition further comprises a phospholipid. In some embodiments, the phospholipid is present in the LF92 lipid composition at a molar percentage of at least about 10%, 15%, 20%, or 25%. In some embodiments, the phospholipid is present in the LF92 lipid composition at a molar percentage of at most about 40%, 35%, or 30%. In some embodiments, the phospholipid is present in the LF92 lipid composition at a molar percentage of about 10%, 15%, 20%, 25%, 30%, 35%, or 40%, or any range between any two of the foregoing. In some embodiments, the phospholipid is present in the LF92 lipid composition at a molar percentage of 10% to 40%, or 20% to 40%. In some embodiments, lipid composition further comprises a steroid or steroid derivative. In some embodiments, the lipid composition further comprises a polymer-conjugated lipid (e.g., poly(ethylene glycol) (PEG)-conjugated lipid).

### SORT Formulations

In another aspect, the lipid composition of the present disclosure comprises (i) an ionizable cationic lipid, (ii) a phospholipid, and (iii) a selective organ targeting (SORT) lipid separate from the ionizable cationic lipid and the phospholipid. In some embodiments, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 5 to about 8. In some embodiments, the ionizable cationic lipid is a dendrimer or dendron. In some embodiments, the ionizable cationic lipid comprises at least two C₆-C₂₄ alkyl or alkenyl groups.

### Phospholipids or Other Zwitterionic Lipids

In various embodiments described herein in the “lipid formulations” section, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

### Steroids or Steroid Derivatives

In various embodiments described herein in the “lipid formulations” section, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

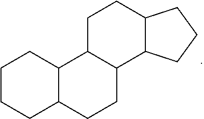 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

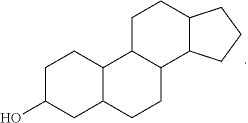 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

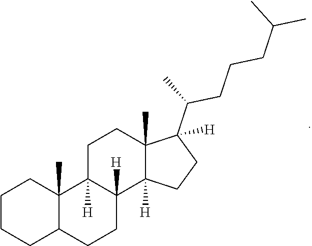

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestene and a sterol or a derivative thereof.

### Polymer-Conjugated Lipids

In various embodiments described herein in the “lipid formulations” section, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In various embodiments of the lipid formulations, the PEG lipid has a structural formula:

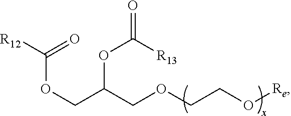

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Re is alkyl(css) such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In various embodiments, the PEG lipid has a structural formula:

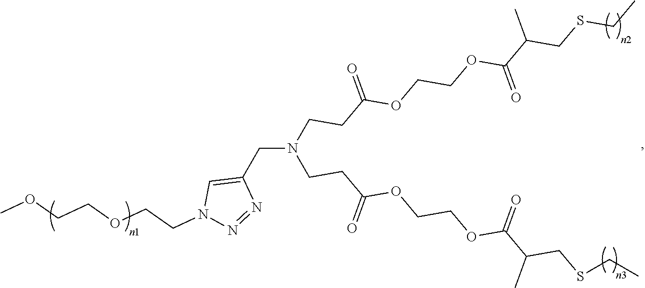

wherein: n₁ is an integer between 1 and 100 and n₂ and n3 are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n3 is from 5 to 23. In some embodiments, n3 is 11 to about 17.

### Pharmaceutical Compositions

Some embodiments of the (e.g., pharmaceutical) composition disclosed herein comprise a particular molar ratio of the components or atoms. In some embodiments, the (e.g., pharmaceutical) composition comprises a particular molar ratio of nitrogen in the lipid composition to the phosphate in the polynucleotide (N/P ratio). In some embodiments, the molar ratio of nitrogen in the lipid composition to phosphate in the polynucleotide (N/P ratio) is no more than about 20:1. In some embodiments, the N/P ratio is from about 5:1 to about 20:1. In some embodiments, the N/P ratio is no more than 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or less. In some embodiments, the N/P ratio is at least 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or more. In some embodiments, the N/P ratio is of any one of the following values or within a range of any two of the following values: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1.

In some embodiments, composition comprises a particular molar ratio of the polynucleotide to total lipids of the lipid composition. In some embodiments, the molar ratio of the polynucleotide to total lipids of the lipid composition is no more than about 1:1, 1:10, 1:50, or 1:100. In some embodiments, the molar ratio of the polynucleotide to total lipids of the lipid composition is no more than about 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:75, or 1:100 or less. In some embodiments, the molar ratio of the polynucleotide to total lipids of the lipid composition is at least about 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:75, or 1:100 or more. In some embodiments, the molar ratio of the polynucleotide to total lipids of the lipid composition is of any one of the following values or within a range of any two of the following values: 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:75, and 1:100.

In some embodiments, the lipid composition comprises a plurality of particles. The plurality of particles may be characterized by a particular size. For example, the plurality of particles may have an average size. In some embodiments the lipid composition comprises a plurality of particles characterized by a size (e.g. average size) of 100 nanometers (nm) or less. The plurality of particles may be characterized by a size of no more than 10 nm, 20 n, 30 nm, 40 nm, 50 n, 60 nm, 70 nm, 80 nm, 90 nm, 100 nm or less. The plurality of particles may be characterized by a size of at least 10 nm, 20 nm, 30 nm, 40 nm, 50 nm, 60 nm, 70 nm, 80 nm, 90 nm, 100 nm or more. The plurality of particles may be characterized by a size of any one of the following values or within a range of any two of the following values: 10 nm, 20 nm, 30 nm, 40 nm, 50 nm, 60 nm, 70 nm, 80 nm, 90 nm, and 100 nm.

In some embodiments, the plurality of particles may be characterized by a particular polydispersity index (PDI) In some embodiments, the lipid composition comprises a plurality of particles characterized by a polydispersity index (PDI) of no more than about 0.2.

In some embodiments, the plurality of particles may be characterized by a particular zeta potential. In some embodiments, the lipid composition comprises a plurality of particles characterized by a negative zeta potential of −5, −4, or −3 millivolts (mV) or a smaller negative value. For example, the plurality of particles may be characterized by a negative zeta potential of −2 mV. In some embodiments, the lipid composition comprises a plurality of particles with a negative zeta potential of −5 millivolts (mV) or less. In some embodiments, the lipid composition comprises a plurality of particles with a negative zeta potential of −10 millivolts (mV) or less. In some embodiments, the lipid composition comprises a plurality of particles with a negative zeta potential of −15 millivolts (mV) or less. In some embodiments, the lipid composition comprises a plurality of particles with a negative zeta potential of −20 millivolts (mV) or less. In some embodiments, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments, the lipid composition comprises a plurality of particles with a negative zeta potential of 15 millivolts (mV) or less. In some embodiments, the lipid composition comprises a plurality of particles with a negative zeta potential of 20 millivolts (mV) or less.

In some embodiments, the lipid composition comprises a plurality of particles with a negative zeta potential of −5 millivolts (mV) or more. In some embodiments, the lipid composition comprises a plurality of particles with a negative zeta potential of −10 millivolts (mV) or more. In some embodiments, the lipid composition comprises a plurality of particles with a negative zeta potential of −15 millivolts (mV) or more. In some embodiments, the lipid composition comprises a plurality of particles with a negative zeta potential of −20 millivolts (mV) or more. In some embodiments, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments, the lipid composition comprises a plurality of particles with a negative zeta potential of 15 millivolts (mV) or more. In some embodiments, the lipid composition comprises a plurality of particles with a negative zeta potential of 20 millivolts (mV) or more.

The particles of the lipid composition may encapsulate other components of the (e.g., pharmaceutical) composition. In some embodiments, the polynucleotide is encapsulated in particles of the lipid composition. In some embodiments, at least about 85% of the polynucleotide is encapsulated in particles of the lipid compositions. In some embodiments, at least about 75% of the polynucleotide is encapsulated in particles of the lipid compositions. In some embodiments, at least about 65% of the polynucleotide is encapsulated in particles of the lipid compositions.

In some embodiments (especially of the SORT formulations), the lipid composition (with or without polynucleotide(s) assembled therewith) comprises particular physical characteristic(s). For example, the lipid composition may comprise an apparent ionization constant (pKa). In some embodiments, the lipid composition has an (pKa) is of about 8 or higher. In some embodiments, the lipid composition has an (pKa) is within a range of 8 to 13. In some embodiments, the lipid composition has an (pKa) is of 13 or less.

In some embodiments, the (e.g., pharmaceutical) composition comprises one or more pharmaceutically acceptable excipients.

In some embodiments, the (e.g., pharmaceutical) composition is formulated for inhalation. In some embodiments, the (e.g., pharmaceutical) composition is able to be aerosolized, nebulized, or in an (e.g. inhalable) aerosol composition. In some embodiments, the present disclosure provides an aerosol composition comprising a (e.g., pharmaceutical) composition as described elsewhere herein.

In some embodiments, the (e.g., pharmaceutical) composition may be a dry powder. The dry powder may comprise a polynucleotide (as described anywhere herein) assembled with a lipid composition (as described anywhere herein). The dry powder may be administered to a subject in the dry powder form. The dry powder may be generated by spray drying. The dry powder formulation may maintain an encapsulation or interaction of the polynucleotide with the lipid composition (e.g., nanoparticle or nanocapsule). In some cases, the (e.g., pharmaceutical) composition may be a dry powder for delivery via inhalation.

In some embodiments, the aerosol composition is generated by a nebulizer. The nebulizer may comprise a nebulization rate from 0.2 milliliter (mL) per minute (mL/min) to 1 mL/min. The nebulization rate may allow a therapeutically effective dose to be administered to the subject. In some embodiments, the aerosol composition is generated by a nebulizer at a nebulization rate of no more than 70 mL/minute. In some embodiments, the aerosol composition is generated by a nebulizer at a nebulization rate of no more than 50 mL/minute. In some embodiments, the aerosol composition is generated by a nebulizer at a nebulization rate of no more than 30 mL/minute.

In some embodiments, the aerosol composition has an average or median droplet size. For example, the average or median droplet size may be from 1 micron (μm) to 10 μm. The average or median droplet size may allow a therapeutically effective dose to be administered to the subject. In some embodiments, the aerosol composition has an average droplet size from about to about 0.5 micron (μm) to about 10 μm. In some embodiments, the aerosol composition has an average droplet size from about to about 0.5 micron (μm) to about 10 μm. In some embodiments, the aerosol composition has an average droplet size from about to about 1 micron (μm) to about 10 μm. In some embodiments, the aerosol composition has an average droplet size from about to about 0.5 micron (μm) to about 5 μm. In some embodiments, the droplet size varies less than about 50% for a duration of about 24 hours under a storage condition. In some embodiments, droplets of the aerosol composition are characterized a polydispersity index (PDI) of no more than about 0.5. In some embodiments, the aerosol droplets are generated by a nebulizer at a nebulization rate of no more than 70 mL/minute. In some embodiments, the aerosol droplets have a mass median aerodynamic diameter (MMAD) from about 0.5 micron (μm) to about 10 μm. In some embodiments, the droplet size varies less than about 50% for a duration of about 24 hours under a storage condition. In some embodiments, droplets of said aerosol composition are characterized by a geometric standard deviation (GSD) of no more than about 3.

In some embodiments, the dose is administered intradermally, subcutaneously, orally, intravenously, intravitreally (or otherwise injected into the eye), intra-arterially, intra-abdominally, intraperitoneally, intrathecally, or intramuscularly. In some embodiments, the (e.g., pharmaceutical) composition is administered using a device implanted into the eye or other body part. In some embodiments, the subject is selected from the group consisting of mouse, rat, monkey, and human.

The (e.g., pharmaceutical) composition can be administered for therapy by any suitable route including oral, rectal, nasal, topical (including transdermal, aerosol, buccal and sublingual), vaginal, parenteral (including subcutaneous, subcutaneous by infusion pump, intramuscular, intravenous and intradermal), intravitreal, and pulmonary. The (e.g., pharmaceutical) composition can take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition, the (e.g., pharmaceutical) composition can also contain other pharmaceutically active compounds or a plurality of compounds of the invention.

In some embodiments, the (e.g., pharmaceutical) composition can be administered subcutaneously, orally, intramuscularly, or intravenously. In one embodiment, the (e.g., pharmaceutical) composition is administered at a therapeutically effective dose. The (e.g., pharmaceutical) composition may be administered via inhalation. For example, the composition may be aerosolizable or inhalable.

In some cases, the administration of a dose may be performed over a duration, e.g., a short period of time. The duration may be no more than 10 minutes (e.g., from about 5 to 8 min). In some embodiments, the administration of a dose of the therapeutic agent may be repeated.

The administration of the compositions may result in a therapeutic effect in the subject or subject's cells, e.g., comparable to normal controls. For example, the cilia of the lungs may recover or improve in their function. A beat frequency and or synchronized (e.g., wave-like) motion of cilia may be recovered or improved in the subject after administration of the compositions described throughout this application. The administration may have minimal off-target or negative byproducts. For example, the administration of the compositions may retain cellular viability throughout the subject.

### Kits

Provided herein, in some embodiments, is a kit comprising a (e.g., pharmaceutical) composition described herein, a container, and a label or package insert on or associated with the container.

### Methods

Provided herein includes a method for enhancing an expression or activity of dynein axonemal intermediate chain 1 (DNAI1) protein in a (e.g., lung) cell, the method comprising: contacting said (e.g., lung) cell with a composition comprising a polynucleotide that encodes a DNAI1 protein (such as described herein), thereby providing a(n) (e.g., therapeutically) effective amount or activity of a functional variant (e.g., wild-type form) of DNAI1 protein in said (e.g., lung) cell.

In some embodiments, the method provides a(n) (e.g., therapeutically) effective amount or activity of said functional variant (e.g., wild-type form) of DNAI1 protein in said (e.g., lung) cell at least about 6 hours after said contacting. The contacting may be in vivo. The contacting may be ex vivo. The contacting may be in vitro. In some embodiments, mucus is present in said contacting.

In some embodiments, the (e.g., lung) cell is in a ciliary axoneme. In some embodiments, the (e.g., lung) cell is an airway epithelial cell (e.g., a bronchial epithelial cell). In some embodiments, the (e.g., lung) cell exhibits a mutation in DNAI1 gene or transcript. In some embodiments, said contacting comprises contacting a plurality of (e.g., lung) cells that comprises said (e.g., lung) cell.

In some embodiments, said contacting is repeated. In some embodiments, said repeated contacting is at least once a week, at least twice a week, or at least three times a week. In some embodiments, at least one contacting steps of said repeated contacting is followed by a treatment holiday. In some embodiments, the said repeated contacting is characterized by a duration of at least 1, 2, or 3 week(s). In some embodiments, mucus is present in one or more contacting steps of said repeated contacting.

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, or 7 days) after said contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The contacting may be ex vivo or in vitro. The change in the ciliary beat activity may include one or more of the following: an increase or decrease in beat frequency that approaches a normal beat frequency (9-17 Hz), improved synchronization, and an increase in area (e.g., %) with ciliary activity.

Ciliary function may be measured by any method known in the art. In some embodiments, ciliary activity is measured by comparing a measured ciliary beat frequency (CBF) to a normal value (e.g., 7-16 Hz). In some embodiments, CBF may be determined by imaging and counting ciliary beating cycles. In some embodiments, the imaging technique may comprise microscopy, tomography, videography, or any combination thereof. In some embodiments, ciliary function may be measured by measurement of ciliary shaft structure. In some embodiments, the measurement may comprise imaging comprising microscopy, tomography, videography, or any combination thereof. In some embodiments, ciliary function may be measured by expression of ciliary proteins. Expression of ciliary proteins may be determined by any technique known in the art (e.g., proteomics, immunofluorescence, western blotting).

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, 7, 8, 9, 10, 11, 12, 13, or 14 days) after a contacting of said repeated contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The repeated contacting(s) may be ex vivo or in vitro. The change in the ciliary beat activity may include one or more of the following: an increase or decrease in beat frequency that approaches a normal beat frequency (9-17 Hz), improved synchronization, and an increase in area (e.g., %) with ciliary activity.

Provided herein includes a method for treating a subject having or suspected of having primary ciliary dyskinesia (PCD), comprising administering to the subject a (e.g., pharmaceutical) composition as provided hereinabove or elsewhere herein. The (e.g., pharmaceutical) compositions as described hereinabove or elsewhere herein may be effective at treating a subject having PCD. The (e.g., pharmaceutical) compositions as described hereinabove or elsewhere herein may be effective at treating a subject suspected of having PCD. The (e.g., pharmaceutical) compositions may alleviate or eliminate symptoms of PCD in the subject (e.g., regardless whether the subject has been determined to have PCD).

Provided herein, in some aspects, is a method for treating a subject having or suspected of having primary ciliary dyskinesia (PCD), comprising administering to the subject a (e.g., pharmaceutical) composition comprising a heterologous polynucleotide assembled with a lipid composition, which heterologous polynucleotide encodes a dynein axonemal intermediate chain 1 (DNAI1) protein, thereby resulting in a heterologous expression of the DNAI1 protein within cells of the subject.

Provided herein method of treating a having or suspected of having primary ciliary dyskinesia (PCD) comprising administrating the . . . (e.g., pharmaceutical) compositions that comprise component, thereby generating an improved efficacy or outcome of treatment. The methods for treating a subject having or suspected of having primary ciliary dyskinesia (PCD) may be more effective at delivery to a particular cell, cell type, organ, or bodily region as compared to a treating with a reference composition or compound. The methods for treating a subject having or suspected of having primary ciliary dyskinesia (PCD) described elsewhere herein may be more effective at generating increase expression of a corresponding polypeptide of a delivered polynucleotide. The methods for treating a subject having or suspected of having primary ciliary dyskinesia (PCD) described elsewhere herein may be more effective at generating a larger number of cells that express a corresponding polypeptide of a delivered polynucleotide. The methods for treating a subject having or suspected of having primary ciliary dyskinesia (PCD) described elsewhere herein may result in an increase uptake of the polynucleotide as compared to a reference polynucleotide. The increased uptake may be result of improved stability of polynucleotide or an improved targeting of the composition to a particular cell type or organ. In some embodiments, the methods for treating a subject having or suspected of having primary ciliary dyskinesia (PCD) comprise administering to the subject a composition comprising a SORT lipid present in an amount in the lipid composition to effect a greater expression or activity of the polynucleotide (or corresponding polypeptide of the polynucleotide) in a cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect at least a 1.1 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 2-fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 5 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 10-fold greater expression or activity of the polynucleotide (or corresponding polypeptide of the polynucleotide) in a cell compared to that achieved with a reference lipid composition comprising LF92, a phospholipid, cholesterol, and a PEG-lipid.

In some embodiments, the methods for treating a subject having or suspected of having primary ciliary dyskinesia (PCD) comprise administering to the subject a composition comprising a SORT lipid present in an amount in the lipid composition to effect an expression or activity of the polynucleotide (or corresponding polypeptide of the polynucleotide) in a greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 1.1-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 2-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 5-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 10-fold greater plurality of cells compared to that achieved with a reference lipid composition comprising LF92, a phospholipid, cholesterol, and a PEG-lipid.

In some embodiments, the methods for treating a subject having or suspected of having primary ciliary dyskinesia (PCD) comprise administering to the subject a composition comprising a SORT lipid present in an amount in the lipid composition to effect an uptake of the polynucleotide in a greater plurality of cells compared to that achieved with a reference lipid composition comprising LF92, a phospholipid, cholesterol, and a PEG-lipid. In some embodiments, the SORT lipid is present in an amount in the lipid composition to effect an uptake of the polynucleotide in a greater amount to a cell compared to that achieved with a reference lipid composition comprising LF92, a phospholipid, cholesterol, and a PEG-lipid.

In some embodiments, the lipid composition is described hereinabove in the “SORT formulations” section. The lipid composition may comprise (i) an ionizable cationic lipid (such as one described hereinabove in the “SORT formulations” section), (ii) a phospholipid (such as one described hereinabove in the “SORT formulations” section), and (iii) a selective organ targeting (SORT) lipid separate from the ionizable cationic lipid and the phospholipid. The SORT lipid may be one described hereinabove in the “Selective Organ Targeting Compounds” section. The ionizable cationic lipid may be one described hereinabove in the “Dendrimers of Formula (I)” or “Dendrimers of Formula (X)” section.

In some other embodiments, the lipid composition is described hereinabove in the “LF92 formulations” section. For example, the lipid composition comprises a cationic lipid having a structural formula (I′):

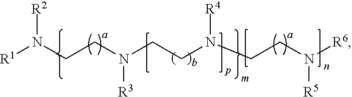

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I′) may be protonated to provide a cationic lipid. In some embodiments, the cationic lipid is 13,16,20-tris(2-hydroxydodecyl)-13,16,20,23-tetraazapentatricontane-11,25-diol. In some embodiments, the cationic lipid is (11R,25R)-13,16,20-tris(®-2-hydroxydodecyl)-13,16,20,23-tetraazapentatricontane-11,25-diol.

In various embodiments of the method described herein in this section, the heterologous polynucleotide may be one described hereinabove in the “Nucleic Acids” section. For example, the polynucleotide may comprise a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15.

The methods for treating the subject may comprise administering using a variety of administration methods as described elsewhere herein. The administration may be performed in such a way to target or come in contact with an organ of interest. In some embodiments, the administering comprises administering to a lung by nebulization.

The methods as described herein may comprises treating or administering a composition to the subject. In some cases, the subject may be determined to have PCD. The subject may be observed or determined to have a genetic or expression profile that is aberrant from a health individual. An aberrant genetic profile or expression profile may be indicative of a particular disease or disorder. The subject may be determined to exhibit aberrant expression or activity of the DNAI1 gene or protein. The subject may have a pathogenic mutation in the DNAI1 gene or protein. The aberrant expression or activity may be an excess or increased activity of a protein or gene that results in a disease state. The aberrant expression or activity may be a decrease or loss of activity of a protein or gene that results in a disease state. The aberrant expression may be a loss of activity such that a particular function of a protein is lost. The aberrant expression may be alleviated by the introduction of a composition that increases the expression of a protein and allows a regain of protein function in a cell or organ. The subject may have a decrease functionality of their lungs. The subject may have a decreased lung capacity or ability to expel air from the lungs. For example, subject may have a lower forced expiratory volume in one second (FEVI) as compared to a healthy or baseline individual. The subject may have a FEVI value of 30% to 90% or 40% to 90%.

The cells comprising aberrant expression and/or the cells wherein the composition are administered to may be a particular type of cell or located in a particular area of the body of the subject. The cells may be lung cells. The cells may be located in the lung of the subject. The cells may be undifferentiated or differentiated. In some embodiments, the cells comprise ciliated cell(s), club cell(s), or basal cell(s), or any combination thereof. In some embodiments, the cells comprise lung epithelial cell(s). In some embodiments, the cells are ciliated cells. In some embodiments, the ciliated cells are ciliated epithelial cells. For example, the ciliated cells may be ciliated airway epithelial cells. In some embodiments, the epithelial cells are undifferentiated. In some embodiments, the epithelial cells are differentiated. The cells may be located in the trachea, bronchi, bronchioles, or other parts of the lung or associated areas.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A pharmaceutical composition comprising a polynucleotide assembled with a lipid composition, wherein:
said polynucleotide (1) encodes a dynein axonemal intermediate chain 1 (DNAI1) protein and (2) comprises a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15; and
said lipid composition comprises a cationic lipid having a structural formula (I′):

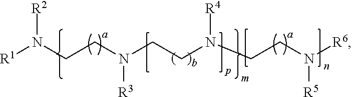

wherein:
p is 1 or 2;
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I′) may be protonated to provide a cationic lipid.

Embodiment 2. The pharmaceutical composition of Embodiment 1, wherein a is 1.

Embodiment 3. The pharmaceutical composition of Embodiment 1 or 2, wherein b is 2.

Embodiment 4. The pharmaceutical composition of any one of Embodiments 1-3, wherein m is 1

Embodiment 5. The pharmaceutical composition of any one of Embodiments 1-4, wherein n is 1.

Embodiment 6. The pharmaceutical composition of any one of Embodiments 1-5, wherein R¹, R², R³, R⁴, R⁵, and R⁶ are each independently H or —CH₂CH(OH)R⁷.

Embodiment 7. The pharmaceutical composition of any one of Embodiments 1-6, wherein R¹, R², R³, R⁴, R⁵, and R⁶ are each independently H or

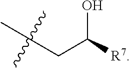

Embodiment 8. The pharmaceutical composition of any one of Embodiments 1-7, wherein R¹, R², R³, R⁴, R⁵, and R⁶ are each independently H or

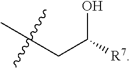

Embodiment 9. The pharmaceutical composition of any one of Embodiments 1-8, wherein R⁷ is C₃-C₁₈ alkyl (e.g., C₆-C₁₂ alkyl).

Embodiment 10. The pharmaceutical composition of any one of Embodiments 1-9, wherein said cationic lipid is 13,16,20-tris(2-hydroxydodecyl)-13,16,20,23-tetraazapentatricontane-11,25-diol:

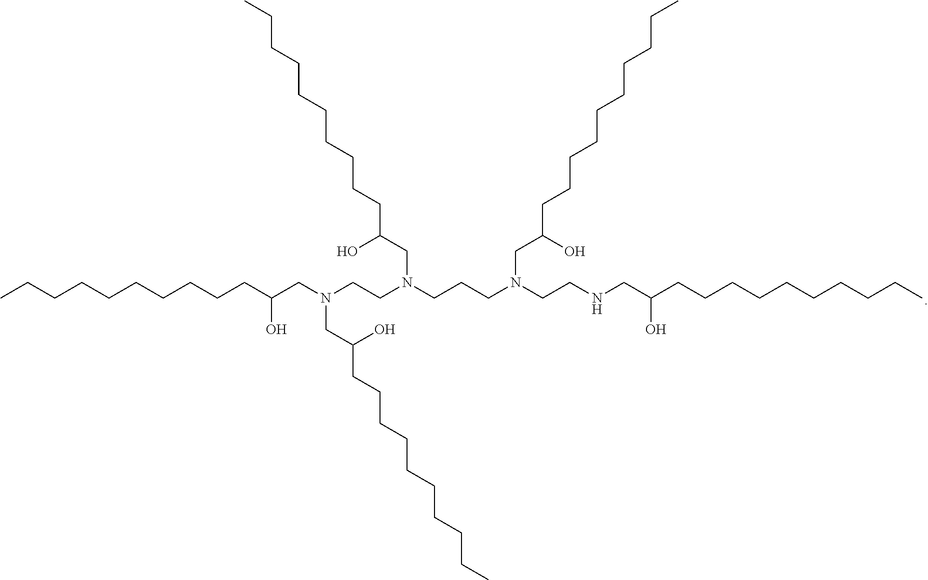 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

Embodiment 11. The pharmaceutical composition of Embodiment 10, wherein said cationic lipid is (11R,25R)-13,16,20-tris(@-2-hydroxydodecyl)-13,16,20,23-tetraazapentatricontane-11,25-diol:

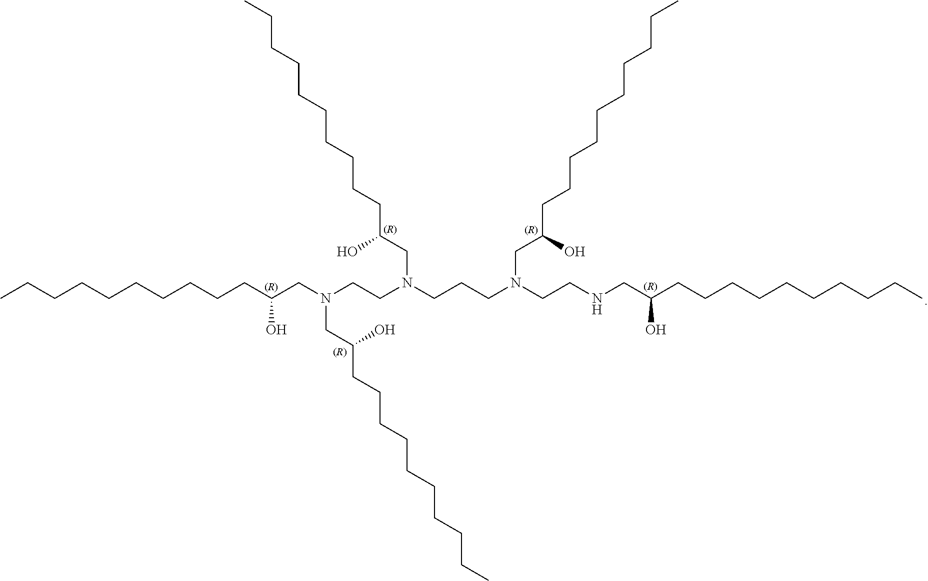

Embodiment 12. The pharmaceutical composition of any one of Embodiments 1-11, wherein said lipid composition comprises said cationic lipid at a molar percentage of no more than 50%.

Embodiment 13. The pharmaceutical composition of any one of Embodiments 1-12, wherein said lipid composition further comprises a phospholipid (e.g., at a molar percentage of at least about 5%).

Embodiment 14. The pharmaceutical composition of any one of Embodiments 1-13, wherein said lipid composition further comprises a steroid or steroid derivative.

Embodiment 15. The pharmaceutical composition of any one of Embodiments 1-14, wherein said lipid composition further comprises a polymer-conjugated lipid (e.g., poly(ethylene glycol) (PEG)-conjugated lipid).

Embodiment 16. The pharmaceutical composition of any one of Embodiments 1-15, wherein said nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotide residues 1 to 1,000) of SEQ ID NO: 15.

Embodiment 17. The pharmaceutical composition of Embodiment 15, wherein said nucleic acid sequence has 100% sequence identity to a sequence over at least 1,000 bases (e.g., nucleotide residues 1 to 1,000) of SEQ ID NO: 15.

Embodiment 18. The pharmaceutical composition of any one of Embodiments 1-17, wherein at least 90%, 95%, or 97% nucleotides replacing uridine within said polynucleotide are nucleotide analogues.

Embodiment 19. The pharmaceutical composition of any one of Embodiments 1-18, wherein fewer than 15% of nucleotides within said polynucleotide are nucleotide analogues.

Embodiment 20. The pharmaceutical composition of any one of Embodiments 1-19, wherein said polynucleotide comprises 1-methylpseudouridine.

Embodiment 21. The pharmaceutical composition of any one of Embodiments 1-20, wherein said nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NO: 16.

Embodiment 22. The pharmaceutical composition of any one of Embodiments 1-21, wherein said nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NO: 16.

Embodiment 23. The pharmaceutical composition of any one of Embodiments 1-22, wherein said nucleic acid sequence comprises fewer codon types encoding an amino acid as compared to a corresponding wild-type sequence selected from SEQ ID NOs: WT1-WT10.

Embodiment 24. The pharmaceutical composition of any one of Embodiments 1-23, wherein at least one type of an isoleucine-encoding codons in said corresponding wild-type sequence is substituted with a synonymous codon type in said nucleic acid sequence.

Embodiment 25. The pharmaceutical composition of any one of Embodiments 1-24, wherein at least one type of a valine-encoding codons in said corresponding wild-type sequence is substituted with a synonymous codon type in said nucleic acid sequence.

Embodiment 26. The pharmaceutical composition of any one of Embodiments 1-25, wherein at least one type of an alanine-encoding codons in said corresponding wild-type sequence is substituted with a synonymous codon type in said nucleic acid sequence.

Embodiment 27. The pharmaceutical composition of any one of Embodiments 1-26, wherein at least one type of a glycine-encoding codons in said corresponding wild-type sequence is substituted with a synonymous codon type in said nucleic acid sequence.

Embodiment 28. The pharmaceutical composition of any one of Embodiments 1-27, wherein at least one type of a proline-encoding codons in said corresponding wild-type sequence is substituted with a synonymous codon type in said nucleic acid sequence.

Embodiment 29. The pharmaceutical composition of any one of Embodiments 1-28, wherein at least one type of a threonine-encoding codons in said corresponding wild-type sequence is substituted with a synonymous codon type in said nucleic acid sequence.

Embodiment 30. The pharmaceutical composition of any one of Embodiments 1-29, wherein at least one type of a leucine-encoding codons in said corresponding wild-type sequence is substituted with a synonymous codon type in said nucleic acid sequence.

Embodiment 31. The pharmaceutical composition of any one of Embodiments 1-30, wherein at least one type of an arginine-encoding codons in said corresponding wild-type sequence is substituted with a synonymous codon type in said nucleic acid sequence.

Embodiment 32. The pharmaceutical composition of any one of Embodiments 1-31, wherein at least one type of a serine-encoding codons in said corresponding wild-type sequence is substituted with a synonymous codon type in said nucleic acid sequence.

Embodiment 33. The pharmaceutical composition of any one of Embodiments 1-32, wherein said pharmaceutical composition comprises an excipient.

Embodiment 34. The pharmaceutical composition of any one of Embodiments 1-32, wherein said polynucleotide is present in said pharmaceutical composition at a concentration of no more than 1 mg/Ml.

Embodiment 35. The pharmaceutical composition of any one of Embodiments 1-34, wherein a molar ratio of nitrogen in said lipid composition to phosphate in said polynucleotide (N/P ratio) is no more than about 20:1.

Embodiment 36. The pharmaceutical composition of Embodiment 35, wherein said N/P ratio is from about 5:1 to about 20:1.

Embodiment 37. The pharmaceutical composition of any one of Embodiments 1-36, wherein a molar ratio of said polynucleotide to total lipids of said lipid composition is no more than about 1:1, 1:10, 1:50, or 1:100.

Embodiment 38. The pharmaceutical composition of any one of Embodiments 1-37, wherein at least about 85% of said polynucleotide is encapsulated in particles of said lipid compositions.

Embodiment 39. The pharmaceutical composition of any one of Embodiments 1-38, wherein said lipid composition comprises particles characterized by a (e.g., average) size of 100 nanometers (n) or less.

Embodiment 40. The pharmaceutical composition of any one of Embodiments 1-39, wherein said lipid composition comprises a plurality of particles characterized by a polydispersity index (PDI) of no more than about 0.2.

Embodiment 41. The pharmaceutical composition of any one of Embodiments 1-40, wherein said lipid composition comprises a plurality of particles characterized by a negative zeta potential of −5, −4, or −3 millivolts (Mv) or less.

Embodiment 42. The pharmaceutical composition of any one of Embodiments 1-41, wherein said pharmaceutical formulation is formulated for inhalation.

Embodiment 43. The pharmaceutical composition of any one of Embodiments 1-42, wherein said pharmaceutical composition is an (e.g., inhalable) aerosol composition.

Embodiment 44. An aerosol composition comprising a pharmaceutical composition of any one of Embodiments 1-42.

Embodiment 45. The pharmaceutical composition of Embodiment 43 or the aerosol composition of Embodiment 44, wherein said aerosol composition is generated by a nebulizer.

Embodiment 46. The pharmaceutical composition of Embodiment 43 or the aerosol composition of Embodiment 44 or 45, wherein said aerosol composition has a (e.g., median, or average) droplet size from 1 micron (μm) to 10 μm.

Embodiment 47. A method for treating a subject having or suspected of having primary ciliary dyskinesia (PCD), comprising administering to said subject a pharmaceutical composition of any one of Embodiments 1-43 and 45-46.

Embodiment 48. A method for treating a subject having or suspected of having primary ciliary dyskinesia (PCD), comprising administering to said subject a pharmaceutical composition comprising a heterologous polynucleotide assembled with a lipid composition, which heterologous polynucleotide encodes a dynein axonemal intermediate chain 1 (DNAI1) protein, thereby resulting in a heterologous expression of said DNAI1 protein within cells of said subject,
wherein said heterologous polynucleotide comprises a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15; and
said lipid composition comprises a cationic lipid having a structural formula (I′):

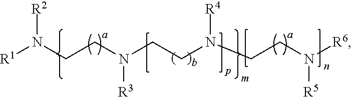

wherein:
p is 1 or 2;
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I′) may be protonated to provide a cationic lipid.

Embodiment 49. The method of Embodiment 47 or 48, wherein a is 1.

Embodiment 50. The method of any one of Embodiments 47-49, wherein b is 2.

Embodiment 51. The method of any one of Embodiments 47-50, wherein m is 1

Embodiment 52. The method of any one of Embodiments 47-51, wherein n is 1.

Embodiment 53. The method of any one of Embodiments 47-52, wherein R¹, R², R³, R⁴, R⁵, and R⁶ are each independently H or —CH₂CH(OH)R⁷.

Embodiment 54. The method of any one of Embodiments 47-53, wherein R¹, R², R³, R⁴, R⁵, and R⁶ are each independently H or

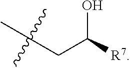

Embodiment 55. The method of any one of Embodiments 47-54, wherein R¹, R², R³, R⁴, R⁵, and R⁶ are each independently H or

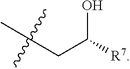

Embodiment 56. The method of any one of Embodiments 47-55, wherein R⁷ is C₃-C₁₈ alkyl (e.g., C₆-C₁₂ alkyl).

Embodiment 57. The method of any one of Embodiments 47-56, wherein said cationic lipid is 13,16,20-tris(2-hydroxydodecyl)-13,16,20,23-tetraazapentatricontane-11,25-diol.

Embodiment 58. The method of Embodiment 57, wherein said cationic lipid is (11R,25R)-13,16,20-tris(®-2-hydroxydodecyl)-13,16,20,23-tetraazapentatricontane-11,25-diol.

Embodiment 59. The method of any one of Embodiments 47-58, wherein said administering comprises administering to a lung by nebulization.

Embodiment 60. The method of any one of Embodiments 47-59, wherein said subject is determined to exhibit an aberrant expression or activity of DNAI1 gene or protein.

Embodiment 61. The method of any one of Embodiments 47-60, wherein said subject is a human.

Embodiment 62. The method of any one of Embodiments 47-61, wherein said cells are in a lung of said subject.

Embodiment 63. The method of Embodiment 62, wherein said cells are ciliated cells.

Embodiment 64. The method of Embodiment 62, wherein said cells are undifferentiated.

Embodiment 65. The method of Embodiment 62, wherein said cells are differentiated.

Embodiment 66. The method of Embodiment 63, wherein said ciliated cells are ciliated epithelial cells (e.g., ciliated airway epithelial cells).

Embodiment 67. The method of Embodiment 66, wherein said ciliated epithelial cells are undifferentiated.

Embodiment 68. The method of Embodiment 66, wherein said ciliated epithelial cells are differentiated.

Embodiment 69. A method for enhancing an expression or activity of dynein axonemal intermediate chain 1 (DNAI1) protein in a (e.g., lung) cell, the method comprising:
contacting said (e.g., lung) cell with a composition comprising a polynucleotide that encodes a DNAI1 protein according to any one of Embodiments 1 to 46,
thereby providing a(n) (e.g., therapeutically) effective amount or activity of a functional variant (e.g., wild-type form) of DNAI1 protein in said (e.g., lung) cell.

Embodiment 70. The method of Embodiment 69, wherein the method provides a(n) (e.g., therapeutically) effective amount or activity of said functional variant (e.g., wild-type form) of DNAI1 protein in said (e.g., lung) cell at least about 6 hours after said contacting.

Embodiment 71. The method of Embodiment 70, wherein said contacting is in vivo.

Embodiment 72. The method of Embodiment 70, wherein said contacting is ex vivo.

Embodiment 73. The method of Embodiment 70, wherein said contacting is in vitro.

Embodiment 74. The method of any one of Embodiments 69-73, wherein said (e.g., lung) cell is in a ciliary axoneme.

Embodiment 75. The method of any one of Embodiments 69-74, wherein mucus is present in said contacting.

Embodiment 76. The method of any one of Embodiments 69-75, wherein said (e.g., lung) cell is an airway epithelial cell (e.g., a bronchial epithelial cell).

Embodiment 77. The method of any one of Embodiments 69-76, wherein said (e.g., lung) cell exhibits a mutation in DNAI1 gene or transcript.

Embodiment 78. The method of any one of Embodiments 69-77, wherein said contacting comprises contacting a plurality of (e.g., lung) cells that comprises said (e.g., lung) cell.

Embodiment 79. The method of any one of Embodiments 69-78, wherein said contacting is repeated.

Embodiment 80. The method of Embodiment 79, wherein said repeated contacting is at least once a week, at least twice a week, or at least three times a week.

Embodiment 81. The method of Embodiment 79 or 80, wherein at least one contacting steps of said repeated contacting is followed by a treatment holiday.

Embodiment 82. The method of any one of Embodiments 79-81, wherein said repeated contacting is characterized by a duration of at least 1, 2, or 3 week(s).

Embodiment 83. The method of any one of Embodiments 79-82, wherein mucus is present in one or more contacting steps of said repeated contacting.

Embodiment 84. The method of any one of Embodiments 69-83, wherein the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, or 7 days) after said contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof.

Embodiment 85. The method of Embodiment 84, wherein said contacting is ex vivo or in vitro.

Embodiment 86. The method of any one of Embodiments 69-85, wherein the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, 7, 8, 9, 10, 11, 12, 13, or 14 days) after a contacting of said repeated contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof.

Embodiment 87. The method of Embodiment 86, wherein said repeated contacting(s) are ex vivo or in vitro.

### EXAMPLES

### Example 1: Preparation of DOTAP or DODAP Modified Lipid Nanoparticles

Lipid nanoparticles (LNPs) are the most efficacious carrier class for in vivo nucleic acid delivery. Historically, effective LNPs are composed of 4 components: an ionizable cationic lipid, zwitterionic phospholipid, cholesterol, and lipid poly(ethylene glycol) (PEG). However, these LNPs result in only general delivery of nucleic acids, rather than organ or tissue targeted delivery. LNPs typically delivery RNAs only to the liver. Therefore, new formulations of LNPs were sought in an effort to provide targeted nucleic acid delivery.

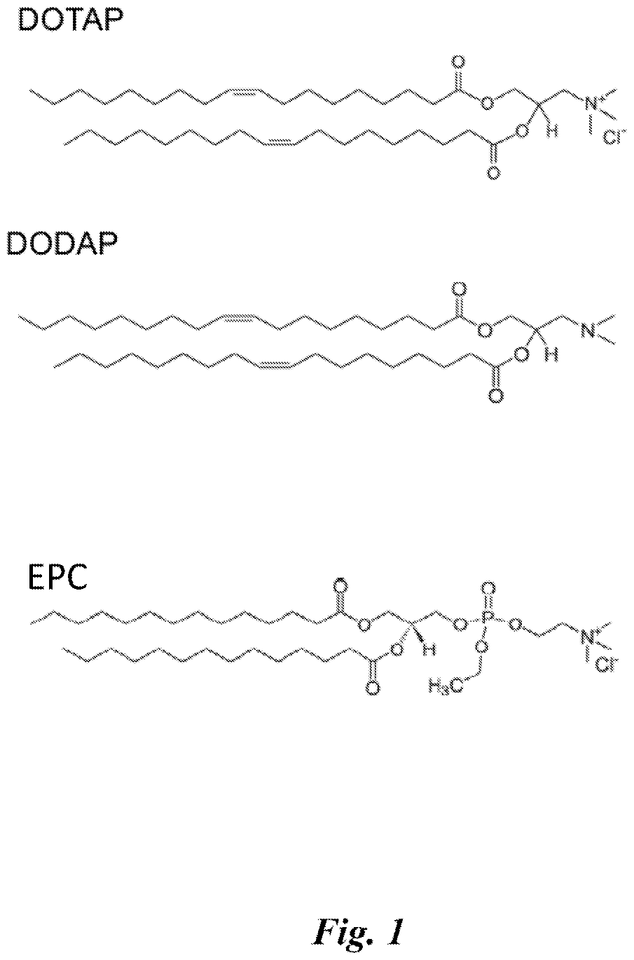

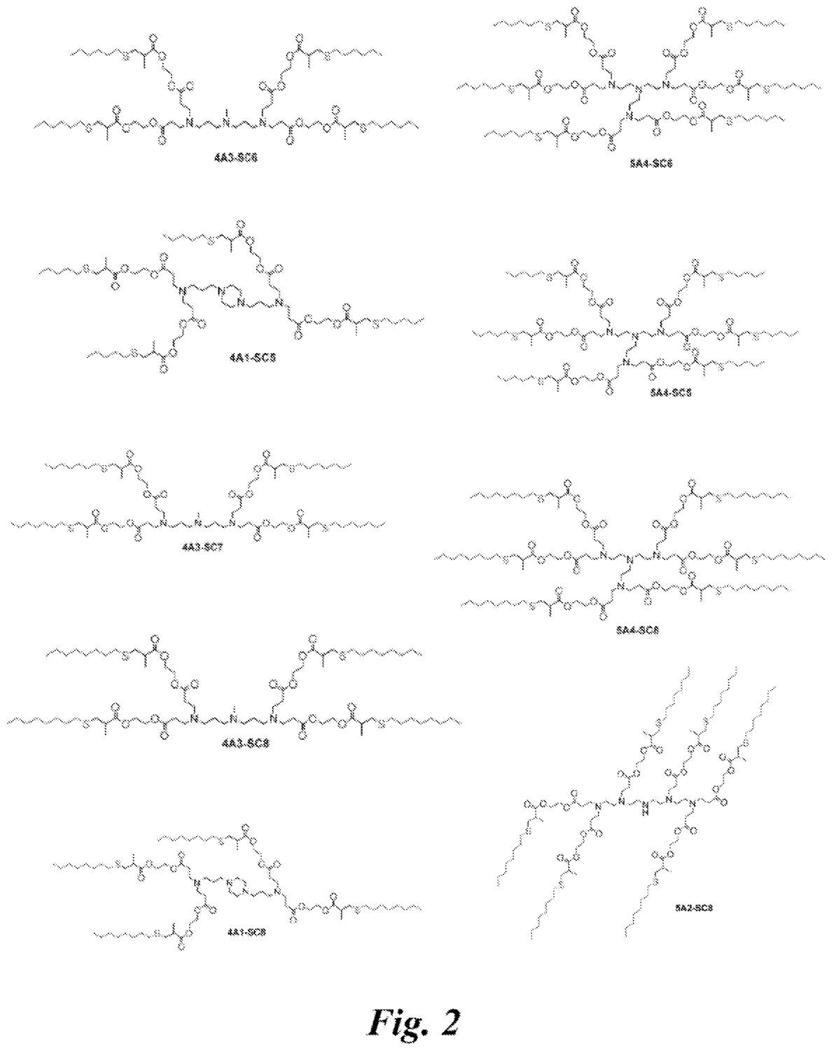

The four canonical types of lipids were mixed in a 15:15:30:3 molar ratio, with or without the addition of a permanently cationic lipid. Briefly, LNPs were prepared by mixing a dendrimer lipid (ionizable cationic), DOPE (zwitterionic), cholesterol, DMG-PEG, and DOTAP (permanently cationic). Alternatively, DOTAP can be substituted for DODAP to generate a LNP comprising DODAP. The structure of DODAP and DODAP are shown in FIG. 1. Various dendrimer lipids that may be used are shown in FIG. 2.

For preparation of the LNP formulation, a dendrimer lipid, DOPE, Cholesterol and DMG-PEG were dissolved in ethanol at desired molar ratios. The mRNA was dissolved in citrate buffer (10 mM, pH 4.0). The mRNA was then diluted into the lipids solution to achieve a weight ratio of 40:1 (total lipids:mRNA) by rapidly mixing the mRNA into the lipids solution at a volume ratio of 3:1 (mRNA:lipids, v/v). This solution was then incubated for 10 min at room temperature. For formation of DOTAP modified LNP formulations, mRNA was dissolved in 1×PBS or citrate buffer (10 mM, pH 4.0), and mixed rapidly into ethanol containing 5A2-SC8, DOPE, Cholesterol, DMG-PEG and DOTAP, fixing the weight ratio of 40:1 (total lipids:mRNA) and volume ratio of 3:1 (mRNA:lipids). Formulation are named X % DOTAP Y (or X % DODAP Y) where X represents the DOTAP (or DODAP) molar percentage in total lipids, and Y represents the type of dendrimer lipid. Alternatively, formulation may be named Y X % DOTAP or Y X % DODAP where X represents the DOTAP (or DODAP) molar percentage in total lipids, and Y represents the type of dendrimer lipid.

### Example 2: SORT LNP Stability

LNPs were tested for stability. 5A2-SC8 20% DODAP (“Liver-SORT) and 5A2-SC8 50% DOTAP (“Lung-SORT”) were generated using either a microfluidic mixing method or a cross/tee mixing method. The different LNP formulations were characterized by size, polydispersity index (PDI) and zeta-potential, were examined by dynamic light scattering, 3 separate times for each formulation. The characteristics of the LNPs are show in Table 7.

TABLE 7
SORT LNP characteristics

| | | | | Encapsulation |
|---|---|---|---|---|
| | Size (nm) | PDI | Zeta (mV) | Efficiency (%) |
| Lung-SORT- | 82.3 | 0.10 | 3.0 | 100 |
| microfluidic | | | | |
| Lung-SORT- | 78.1 | 0.09 | 2.2 | 100 |
| cross/tee mixing | | | | |
| Liver-SORT- | 59.1 | 0.10 | −2.3 | 97 |
| microfluidic | | | | |
| Liver-SORT- | 60.0 | 0.11 | −30 | 96 |
| cross/tee mixing | | | | |

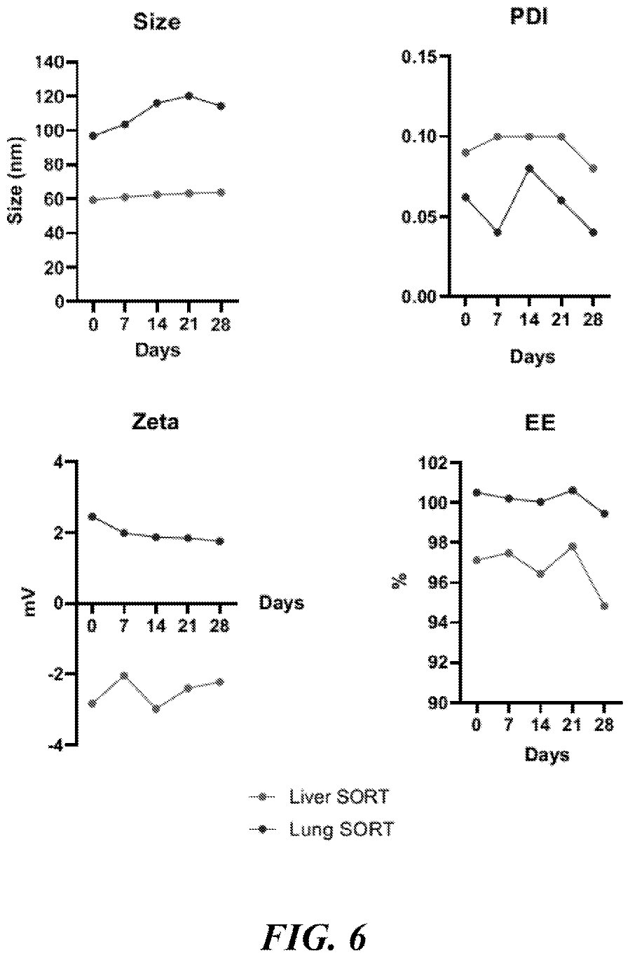

The encapsulation efficiency was tested using a Ribogreen RNA assay (Zhao et al., 2016). Briefly, mRNA was encapsulated with >95% efficiency in LNPs when the mRNA was dissolved in acidic buffer (10 mM citrate, pH 4). The characteristics were observed over 28 days for the two types of LNPs (5A2-SC8 20% DODAP (“Liver-SORT) and 5A2-SC8 50% DOTAP (“Lung-SORT”)). FIG. 6 shows the changes of the characteristics of the LNP over the course of 28 days.

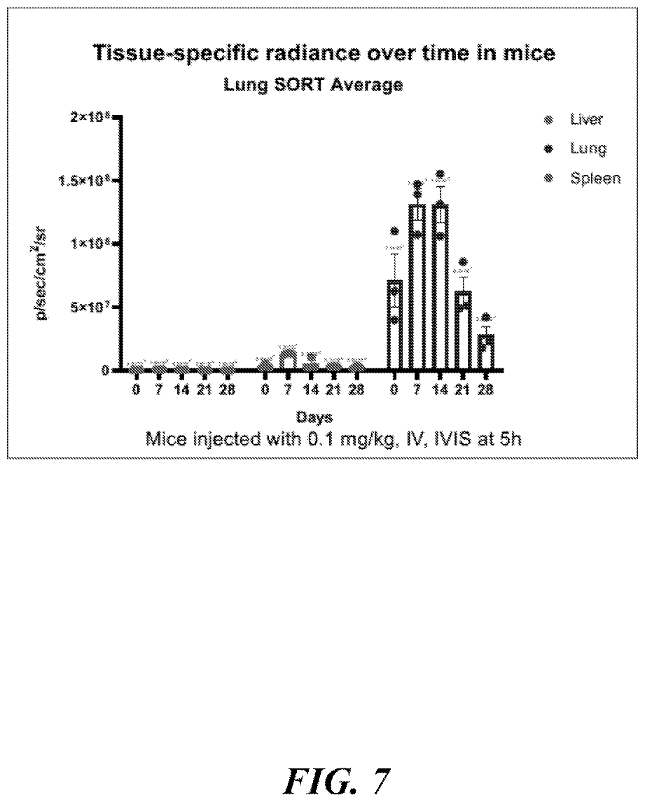

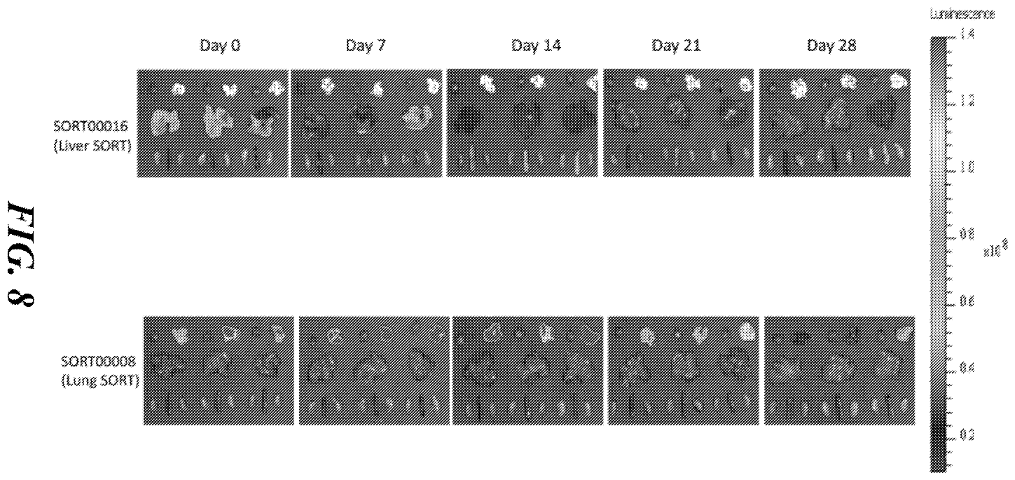

In addition, to the measure of the stability of the LNPs in solution, the stability of the LNPs and resulting mRNA expression was observed in mice. Briefly, mice were injected intravenously with 0.1 mg/kg and observed in vivo. Luciferin was added 5 hrs. after injection and visualized. As shown in FIG. 7, the Lung-SORT LNP generated tissue specific radiance in the lungs which remained high even after 14 days with a slight decay in signal by the 21^{st} and 28^{th} day. FIG. 8 shows images of the organs of the mouse at specific times periods after treated with Lung-SORT or Liver-SORT.

### Example 3: Expression of TR mRNA in Different Cell Types

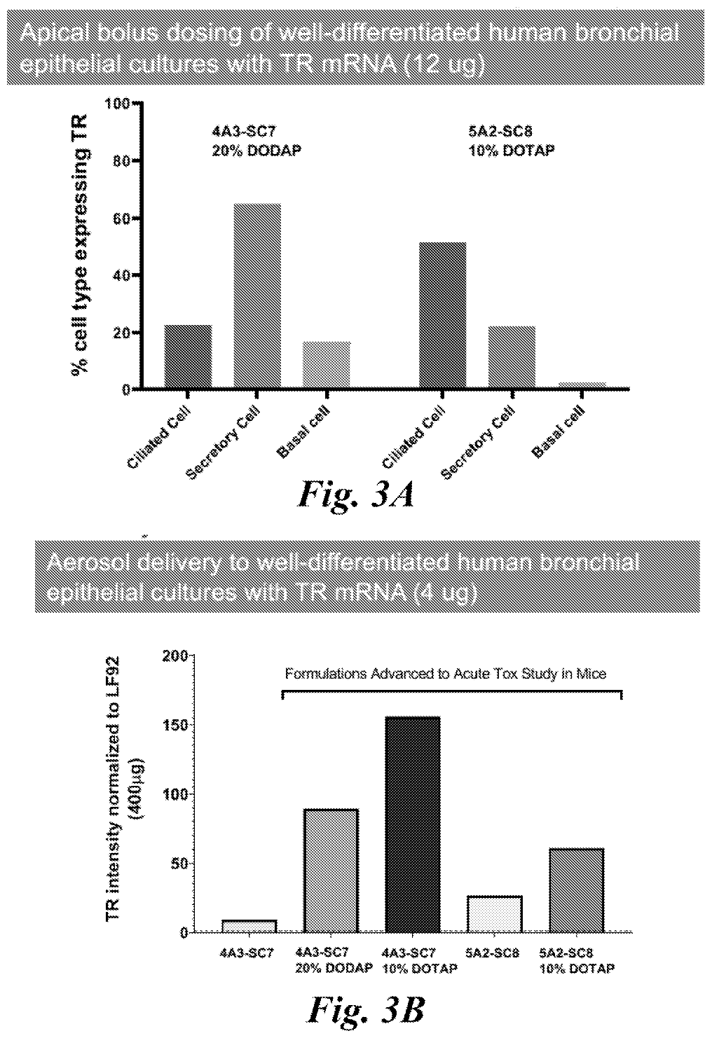

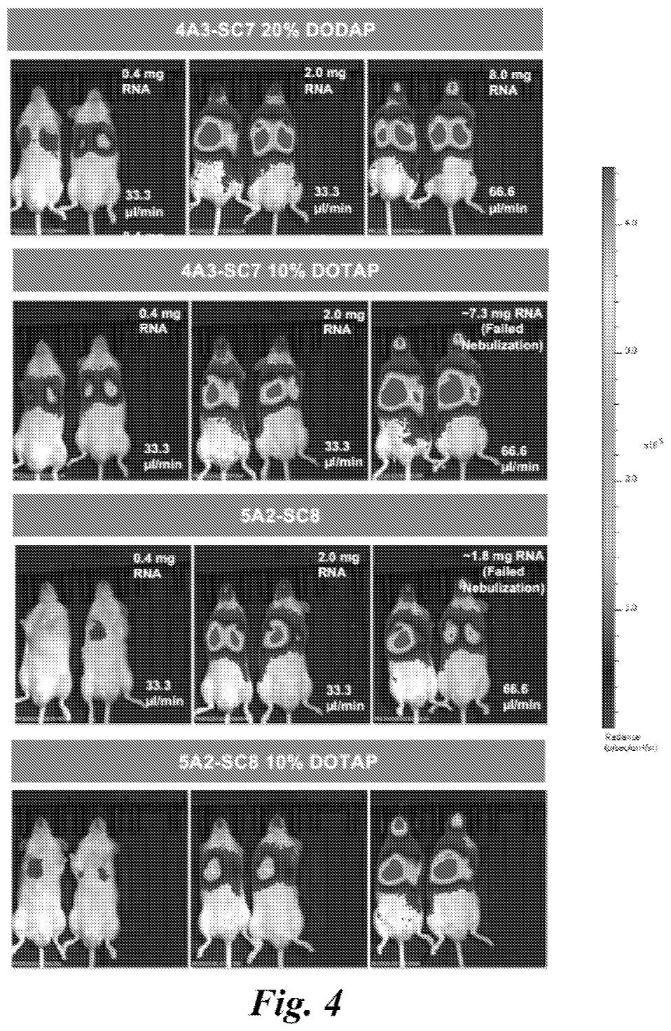

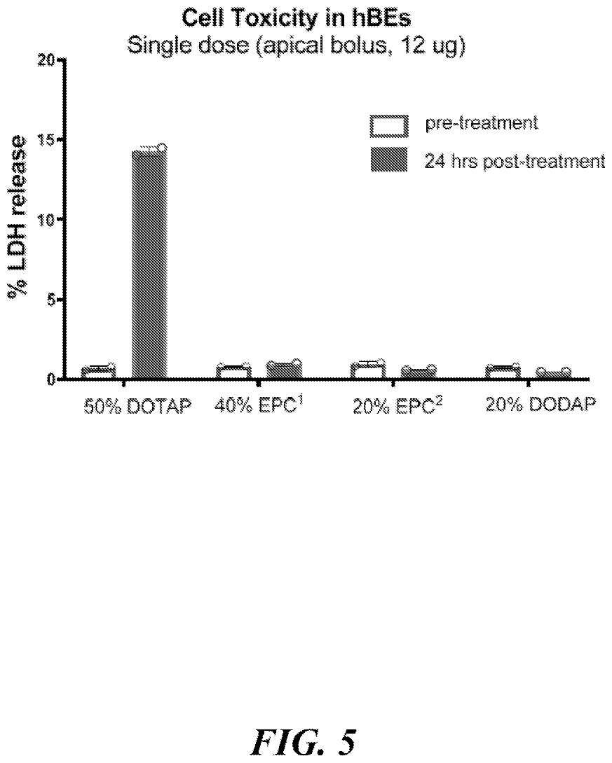

TR mRNA was loaded into either 20% DODAP 4A3-SC7 LNP or 10% DOTAP 5A2-SC8 LNPs and delivered into well-differentiated human bronchial epithelial cultures using apical bolus dosing. Cell expression was observed in various cell type and the percent of the cell type that expressed TR was plotted. As shown in the top panel of FIG. 3, the 20% DODAP 4A3-SC7 LNPs preferentially caused secretory cells to express TR, while 10% DOTAP 5A2-SC8 LNPs cause the ciliated cells to preferentially express TR. This preferential delivery may allow a treatment delivered to the lungs to preferentially affect a specific cell type in the lungs. The TR mRNA was also loaded into LNPs without the SORT lipid (e.g. DODAP or DOTAP) to identify how the DODAP or DOTAP affected the potency. As shown in the bottom panel of FIG. 5, the LNPs comprising DOTAP or DODAP showed increase TR expression compared to their corresponding LNP without DOTAP or DODAP.

### Example 4: Luciferase Activity and Histopathology from LNPs Delivered Via Inhaled Aerosol

Luc mRNA was loaded into a number of LNPs including LNPs of comprising a SORT lipid and a dendrimer. LNPs of 4A3-SC7 20% DODAP, 4A3-SC7 10% DODAP, 5A2-SC8, 5A2-SC8 10% DOTAP were generated and loaded with Luc mRNA. 0.4/2/8 mg of LNP-formulatedLuc2 mRNA (1 mg/ml) was delivered into the pie chamber by nebulization (Aerogen solo), with an estimated (not measured) per mouse delivered dose of 0.01, 0.06 or 0.22 mg/kg. The mice were 7-week-old B6 male albino mice. Luciferin was administered to the mice 5 hrs. after delivery of the LNPs. The luciferase activity was detected as a measure of delivery to a target. FIG. 4 shows the distribution and expression of the luciferase in the mice demonstrating the expression was successful and delivery of the LNPs could be performed using inhaled aerosol delivery.

### Example 5: Toxicity of EPC Containing LNPs

LNPs comprising ethylphosphocholine (EPC) in place of DOTAP or DODAP were tested for toxicity by using apical bolus dosing on human bronchial epithelial cells. The % of lactate dehydrogenase (LDH) that was released was used as a metric of cellular death and indicative of the toxicity of the LNP. The release of LDH was detected prior to treatment (pre-treatment) and 24 post treatment. As shown in FIG. 5, the treatment of 50% DOTAP LNP resulted in an ˜15% LDH release whereas EPC didn't show a significant % LDH release. Importantly, DOTAP and EPC have a similar quaternary amine moiety, indicating that the activity for cell targeting may be similar, but that EPC is considerably less toxic.

### Example 6: Production of DNAI1 mRNA

DNA corresponding to the gene of DNAI1 was synthesized at GenScript. pUC57/DNAI1 was digested with HindIII and EcoRI HF restriction enzymes. Moreover, a digested pVAX120 vector and DNAI1 cDNA were gel purified and ligated (the ORF for DNAI1 is codon optimized). Standard in vitro translation procedure was used for RNA production utilizing unmodified nucleotides. Capping reaction was carried out using Vaccinia Virus capping system and cap 2′-O-methyl transferase.

### Example 7: Detection of DNAI1 mRNA Delivery to a Subject

A subject having or suspected of having primary ciliary dyskinesia (PCD) is given a treatment by administering a composition as described elsewhere herein. The subject is monitored at regular intervals for expression of DNAI1 in the lungs. A sample of lung tissue from the subject is taken comprising ciliated cells of the lung. The cells are harvested and prepared for RNA isolation. cDNA is produced from the RNA using a first strand synthesis kit and random hexamer. qPCR reactions are run using a set of forward and reverse primers and a fluorescent probe, specific to DNAI1 and a second set specific to a control or housekeeping gene for expression normalization. Expression of DNAI1 is detected using a fluorescent readout corresponding the DNAI1 probe.

### Example 8: Prolonged Rescue of Ciliary Activity in KO-Primary Tracheal Mouse ALI Cultures

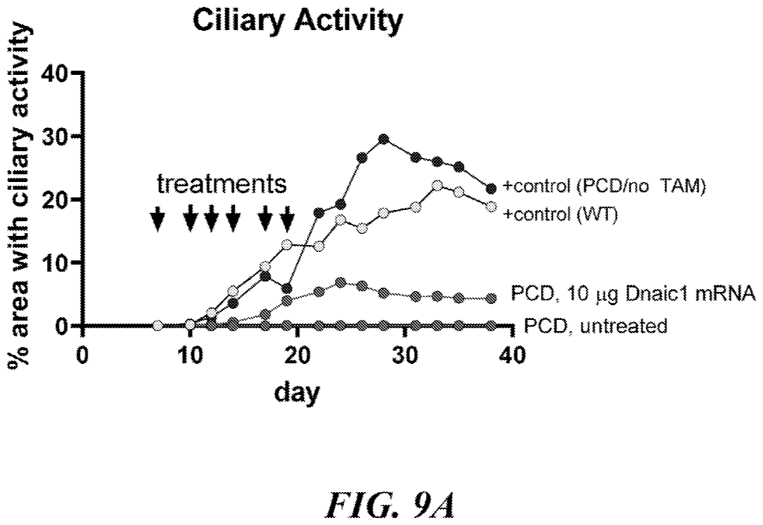

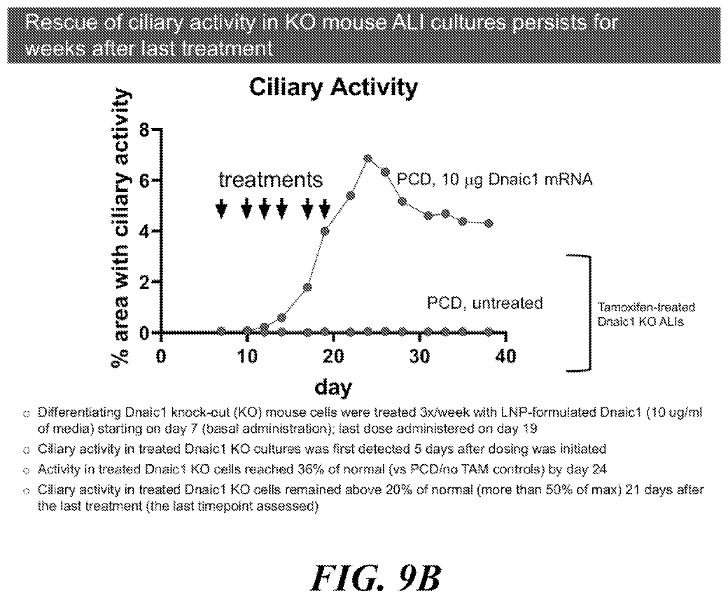

Mouse tracheal epithelial cells (MTEC) were harvested from floxed Dnaic1 mice. The cells were cultured at an air-liquid interface (ALI) and grown until differentiating. The differentiating Dnaic1 knock-out (KO) mouse cells were treated with a low dose of a lipid composition comprising a lipid composition disclosed herein and carrying a DNAI1 mRNA payload. Ciliary activity as measured by ciliary cross-sectional area (CSA) and ciliary beat frequency (CSF) was determined at certain timepoints. Wild type (WT) and PCD/no TAM cells were used as positive controls and untreated Dnaic1 KO cells as negative controls. FIG. 9 demonstrates that ciliary activity in treated Dnaic1 KO cells was higher than untreated Dnaic1 Ko cells. Further, ciliary activity in treated Dnaic1 KO cells persisted past the final treatment.

### Example 9: Detection of DNAI1-HA mRNA Expression in Cells

Human DNAI1 knock-down cells are cultured at an ALI. The cultured cells are treated with a single dose of LNPs containing DNAI1-HA mRNA. Cells are immunostained with anti-acetylated tubulin (ciliated cell marker) and anti-HA antibodies 24 hours, 48 hours, 7 days, and 14 days after dosing. Immunofluorescence imaging of these cells at certain timepoints demonstrates targeted cells successfully express the DNAI1-HA mRNA. Integration of DNAI1-HA into axoneme of cilia is seen to peak between 48-72 hours after treatment.

### Example 10: Aerosol Administration of Formulated DNAI1 mRNA Rescues Ciliary Activity in Knock-Down Primary Bronchial Human ALI Cultures

Human primary bronchial epithelial DNAI1 knock-down cells are cultured at an ALI. Once well-differentiated, cells are treated multiple times per week with a lipid composition as described herein starting on day 25 post ALI culture age. The last does is administered on day 50 post ALI culture. Ciliary activity is by CSA and beat frequency following certain doses. Increased ciliary activity in treated DNAI1 knock-down cultures is detected and persists following the end of treatment.

### Example 11: Preparation of Lipid Composition(s) that Comprise LF92

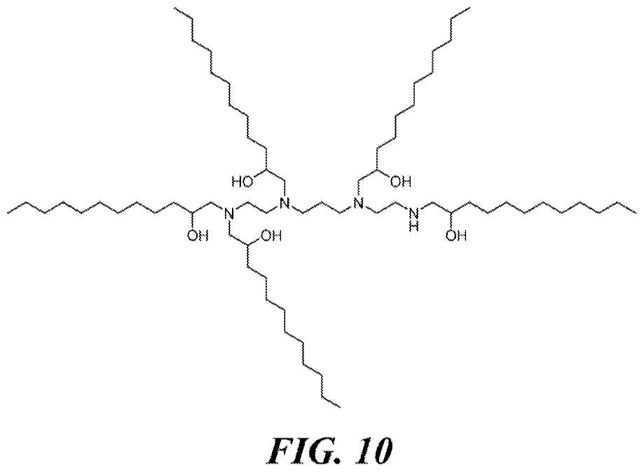

Lipid composition(s) comprising LF92 (see FIG. 10) are prepared according to description of Example 1 of the present application.

### Example 12: Detection of DNAI1 mRNA Delivery to a Subject

A subject having or suspected of having primary ciliary dyskinesia (PCD) is given a treatment by administering an example lipid composition comprising LF92 (as described herein). The subject is monitored at regular intervals for expression of DNAI1 in the lungs. A sample of lung tissue from the subject is taken comprising ciliated cells of the lung. The cells are harvested and prepared for RNA isolation. cDNA is produced from the RNA using a first strand synthesis kit and random hexamer. qPCR reactions are run using a set of forward and reverse primers and a fluorescent probe, specific to DNAI1 and a second set specific to a control or housekeeping gene for expression normalization. Expression of DNAI1 is detected using a fluorescent readout corresponding the DNAI1 probe.

### Example 13: Prolonged Rescue of Ciliary Activity in KO-Primary Tracheal Mouse ALI Cultures

Mouse tracheal epithelial cells (MTEC) are harvested from floxed DNAIC1 mice. The cells are cultured at an air-liquid interface (ALI) and grown until differentiating. The differentiating DNAIC1 knock-out (KO) mouse cells are treated with a low dose of an example lipid composition comprising LF92 (as described herein) and carrying a DNAI1 mRNA payload. Ciliary activity as measured by ciliary cross-sectional area (CSA) and ciliary beat frequency (CSF) is determined at certain timepoints. Wild type (WT) and PCD/no TAM cells are used as positive controls and untreated DNAIC1 KO cells as negative controls.

### Example 14: Detection of DNAI1-HA mRNA Expression in Cells

Human DNAI1 knock-down cells are cultured at an ALI. The cultured cells are treated with a single dose of an example lipid composition comprising LF92 (as described herein) containing DNAI1-HA mRNA. Cells are immunostained with anti-acetylated tubulin (ciliated cell marker) and anti-HA antibodies 24 hours, 48 hours, 7 days, and 14 days after dosing. Immunofluorescence imaging of these cells at certain timepoints demonstrates targeted cells successfully express the DNAI1-HA mRNA. Integration of DNAI1-HA into axoneme of cilia is observed.

### Example 15: Aerosol Administration of Formulated DNAI1 mRNA Rescues Ciliary Activity in Knock-Down Primary Bronchial Human ALI Cultures

Human primary bronchial epithelial DNAI1 knock-down cells are cultured at an ALI. Once well-differentiated, cells are treated multiple times per week with an example lipid composition comprising LF92 (as described herein). Increased ciliary activity in treated DNAI1 knock-down cultures is detected and persists following the end of treatment.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-47. (canceled)

48. A method for treating a subject having or suspected of having primary ciliary dyskinesia (PCD), comprising administering to said subject a pharmaceutical composition comprising a heterologous polynucleotide assembled with a lipid composition, which heterologous polynucleotide encodes a dynein axonemal intermediate chain 1 (DNAI1) protein, thereby resulting in a heterologous expression of said DNAI1 protein within cells of said subject,
wherein said heterologous polynucleotide comprises a nucleic acid sequence having at least about 70% sequence identity to a sequence over at least 1,000 bases of SEQ ID NO: 15; and
wherein said lipid composition comprises a cationic lipid having a structural formula (I′): 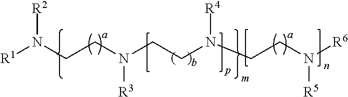
wherein:
p is 1 or 2;
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and R¹, R², R³, R⁴, R⁵, and R⁶ are each independently selected from the group consisting of I, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a polyethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I) may be protonated to provide a cationic lipid.

49. The method of claim 48, wherein a is 1, b is 2, m is 1, and/or n is 1.

50.-52. (canceled)

53. The method of claim 48, wherein R¹, R², R³, R⁴, R⁵, and R⁶ are each independently H, —CH₂CH(OH)R⁷,

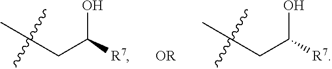

54.-55. (canceled)

56. The method of claim 48, wherein R⁷ is C₃-C₁₈ alkyl or C₆-C₁₂ alkyl.

57. The method of claim 48, wherein said cationic lipid is 13,16,20-tris(2-hydroxydodecyl)-13,16,20,23-tetraazapentatricontane-11,25-diol:

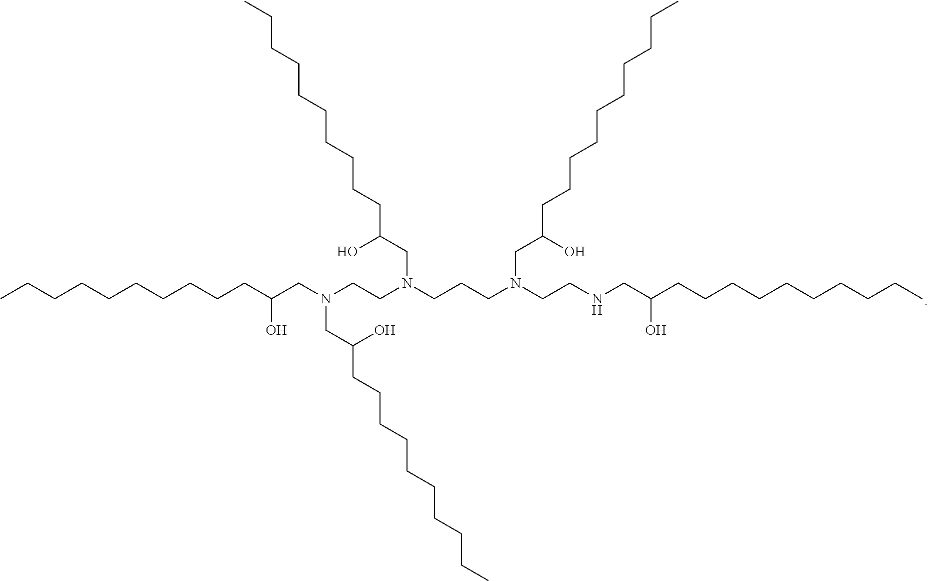 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

58. The method of claim 48, wherein said cationic lipid is (11R,25R)-13,16,20-tris((R)-2-hydroxydodecyl)-13,16 20,23-tetraazapentatricontane-11,25-diol:

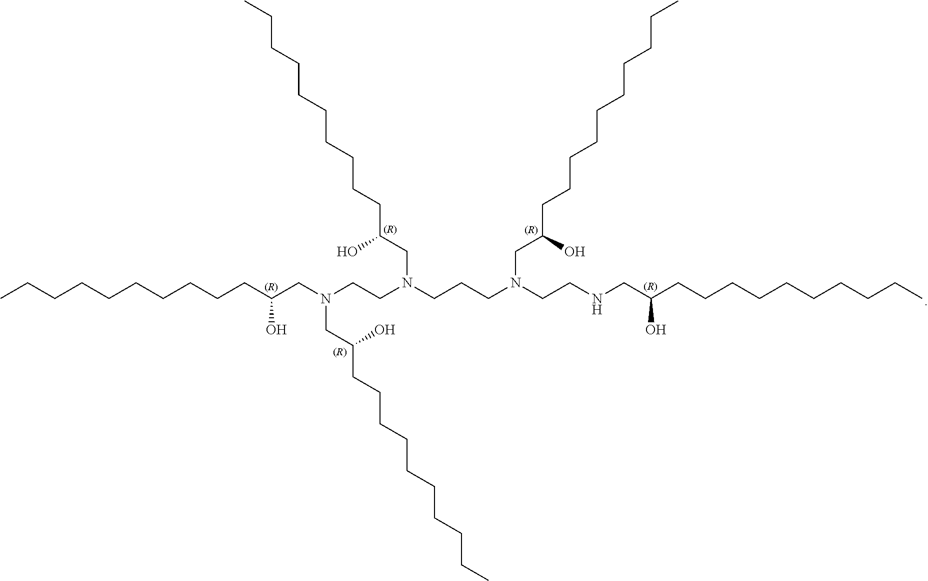

59. The method of claim 48, wherein said administering comprises administering to a lung by nebulization.

60. The method of claim 48, wherein said subject is determined to exhibit an aberrant expression or activity of DNAI1 gene, transcript, or protein.

61. The method of claim 48, wherein said subject is a human.

62. The method of claim 48, wherein said cells are in a lung of said subject.

63. The method of claim 62, wherein said cells are ciliated cells.

64. The method of claim 62, wherein said cells are undifferentiated or are differentiated.

65. (canceled)

66. The method of claim 63, wherein said ciliated cells are ciliated epithelial cells.

67.-68. (canceled)

69. A method for enhancing an expression or activity of dynein axonemal intermediate chain 1 (DNAI1) protein in a cell, the method comprising:
contacting said cell with a composition comprising a polynucleotide that encodes a DNAI1 protein
said polynucleotide comprising a nucleic acid sequence having at least about 70% sequence identity to a sequence over at least 1,000 bases of SEQ ID NO: 15; and
said lipid composition comprises a cationic lipid having a structural formula (I′): 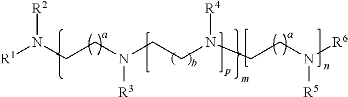
wherein:
p is 1 or 2:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R₇, wherein R⁷ is independently selected from C₃-C₁₈ alkyl, C₃-C₁₈ alkenyl having one C═C double bond, a protecting group for an amino group, —C(═NH)NH₂, a polyethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R₇, wherein R⁷ is independently selected from C₃-C₁₈ alkyl or C₃-C₁₈ alkenyl having one C═C double bond; and wherein one or more of the nitrogen atoms indicated in formula (I′) may be protonated to provide a cationic lipid,
thereby providing a therapeutically effective amount or activity of a functional DNAI1 protein in said cell.

70. The method of claim 69, wherein the method provides a therapeutically effective amount or activity of said functional DNAI1 protein in said cell at least about 6 hours after said contacting.

71. The method of claim 70, wherein said contacting is in vivo, ex vivo, or in vitro.

72.-76. (canceled)

77. The method of claim 69, wherein said cell exhibits a mutation in DNAI1 gene, transcript, or protein.

78. The method of claim 69, wherein said contacting comprises contacting a plurality of cells.

79. The method of claim 69, wherein said contacting is repeated.

80.-83. (canceled)

84. The method of claim 69, wherein the method provides a therapeutically effective amount or activity of said functional DNAI1 protein in said cell at least about 6 24, 48, or 72 hours or at least about 3, 4, 5, 6, or 7 days) after said contacting.

85.-87. (canceled)


SEQUENCE LISTING (17 sequences)

SEQ ID NO: 1 - DNA, 72 bp, Artificial Sequence
  Synthetic Nucleotide
gggagacataaaccctggcgcgctcgcggcccggcactcttctggtccccacagactcagagagaagccacc

SEQ ID NO: 2 - DNA, 72 bp, ARTIFICIAL SEQUENCE
  Synthetic Nucleotide
gggagacataaaccctggcgcgctcgcgggccggcactcttctggtccccacagactcagagagaagccacc

SEQ ID NO: 3 - DNA, 43 bp, ARTIFICIAL SEQUENCE
  Synthetic Nucleotide
gggagactcttctggtccccacagactcagagagaacgccacc

SEQ ID NO: 4 - DNA, 511 bp, ARTIFICIAL SEQUENCE
  Synthetic Nucleotide
gttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatggccacaacc

SEQ ID NO: 5 - DNA, 143 bp, ARTIFICIAL SEQUENCE
  Synthetic Nucleotide
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 6 - DNA, 47 bp, ARTIFICIAL SEQUENCE
  Synthetic Nucleotide
gggagacaagagagaaaagaagagcaagaagaaatataagagccacc

SEQ ID NO: 7 - DNA, 66 bp, ARTIFICIAL SEQUENCE
  Synthetic Nucleotide
gggagacccaagctggctagcgtttaaacttaagcttggcaatccggtactgttggtaaagccacc

SEQ ID NO: 8 - DNA, 291 bp, ARTIFICIAL SEQUENCE
  Synthetic Nucleotide
gggagacccaagctggctagcgtttaaacttaagctttcctttccgggccggctgggcgcgccgaagcgcctgcgccttggctgctggtcggttgctgggtaaccgcgtcagggagttggattctatcctgcaagggcacggggacccacaacgacggctgtccctaaagaaccgttgcgactggtaactgaagtggaagagagtccagatttcttgtgtgtggtcaaggagacggacaaactttttgtcttcagacgagggagcgttttgtaggctctccaggggttgag

SEQ ID NO: 9 - DNA, 186 bp, ARTIFICIAL SEQUENCE
  Synthetic Nucleotide
ggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccattgtcttc

SEQ ID NO: 10 - DNA, 114 bp, ARTIFICIAL SEQUENCE
  Synthetic Nucleotide
tcagtagggtcatgaaggtttttcttttcctgagaaaacaacacgtattgttttctcaggttttgctttttggcctttttctagcttaaaaaaaaaaaaagcaaaattgtcttc

SEQ ID NO: 11 - DNA, 112 bp, ARTIFICIAL SEQUENCE
  Synthetic Nucleotide
tcagtagggttgtaaaggtttttcttttcctgagaaaacaaccttttgttttctcaggttttgctttttggcctttccctagctttaaaaaaaaaaaagcaaaattgtcttc

SEQ ID NO: 12 - DNA, 68 bp, ARTIFICIAL SEQUENCE
  Synthetic Nucleotide
gaagtggcggttcggccggaggttccatcgtatccaaaaggctcttttcagagccacccattgtcttc

SEQ ID NO: 13 - DNA, 239 bp, ARTIFICIAL SEQUENCE
  Synthetic Nucleotide
ggggctggcctcagtctctgtcccatcgcttgaatacagtactcctagggcttgaccctggtacccagcccagccttagcacccagcatgtgaccccactcctgatcaggtcccagcatcttcccttcttgttctgttccttaaggtcccagcaccttaccccaggacttggtcttcaaccaccattacccctctaactttgcacaaataaacctgtgtagaaacccaccccaaaaaaa

SEQ ID NO: 14 - DNA, 66 bp, ARTIFICIAL SEQUENCE
  Synthetic Nucleotide
gggagacccaagctggctagcgtttaaacttcagcttggcaatccggtactgttggtaaagccacc

SEQ ID NO: 15 - DNA, 2100 bp, ARTIFICIAL SEQUENCE
  Synthetic Nucleotide
atgatcccagcaagcgccaaggcaccacacaagcagccccacaagcagagcatcagcatcggcaggggcacaaggaagagggacgaggacagcggaaccgaagtgggagagggaacagacgagtgggcacagagcaaggcaaccgtgcgcccacccgaccagctggagctgacagacgccgagctgaaggaggagttcaccaggatcctgacagccaacaacccacacgccccccagaacatcgtgcgctacagcttcaaggagggcacatacaagccaatcggcttcgtgaaccagctggccgtgcactacacccaagtgggcaacctgatccccaaggacagcgacgagggccggagacagcactacagggacgagctggtggcaggaagccaggagagcgtgaaagtgatcagcgagaccggcaacctggaggaggacgaggagccaaaggagctggagaccgagccaggaagccagacagacgtgcccgcagcaggagcagcagagaaggtgaccgaggaggagctgatgacacccaagcagccaaaggagcggaagctgaccaaccagttcaacttcagcgagagagccagccagacatacaacaacccagtgcgggacagagagtgccagaccgagccaccccccagaaccaacttcagcgccacagccaaccagtgggagatctacgacgcctacgtggaggagctggagaagcaggagaagaccaaggagaaggagaaggccaagacacccgtggccaagaagagcggcaagatggccatgcggaagctgaccagcatggagagccagacagacgacctgatcaagctgagccaggccgccaagatcatggagagaatggtgaaccagaacacctacgacgacatcgcccaggacttcaagtactacgacgacgcagcagacgagtacagggaccaagtgggcacactgctgcccctgtggaagttccagaacgacaaggccaagaggctgagcgtgaccgccctgtgctggaacccaaagtacagggacctgttcgcagtgggatacggaagctacgacttcatgaagcagagcagaggcatgctgctgctgtacagcctgaagaaccccagcttccccgagtacatgttcagcagcaacagcggcgtgatgtgcctggacatccacgtggaccacccctacctggtggccgtgggccactacgacggcaacgtggccatctacaacctgaagaagccccacagccagcccagcttctgcagcagcgccaagagcggcaagcacagcgaccccgtgtggcaggtgaagtggcagaaggacgacatggaccagaacctgaacttcttcagcgtgagcagcgacggcaggatcgtgagctggaccctggtgaagcgcaagctggtgcacatcgacgtgatcaagctgaaggtggagggcagcaccacagaggtgccagagggactgcagctgcacccagtgggatgcggcacagccttcgacttccacaaggagatcgactacatgttcctggtgggcaccgaggagggcaagatctacaagtgcagcaagagctacagcagccagttcctggacacatacgacgcccacaacatgagcgtggacaccgtgagctggaacccctaccacacaaaggtgttcatgagctgcagcagcgactggaccgtgaagatctgggaccacaccatcaagacacccatgttcatctacgacctgaacagcgccgtgggcgacgtggcatgggcaccatacagcagcacagtgttcgcagcagtgaccacagacggcaaggcacacatcttcgacctggccatcaacaagtacgaggccatctgcaaccagcccgtggccgccaagaagaacaggctgacccacgtgcagttcaacctgatccaccccatcatcatcgtgggcgacgaccggggccacatcatcagcctgaagctgagccccaacctgagaaagatgcccaaggagaagaagggacaggaggtgcagaagggaccagcagtggagatcgcaaagctggacaagctgctgaacctggtgcgcgaggtgaagatcaagacctga

SEQ ID NO: 16 - DNA, 2100 bp, ARTIFICIAL SEQUENCE
  Synthetic Nucleotide
atgattcctgcttctgcgaaggctccccataaacagcctcataagcagagcatcagcataggcagaggaaccaggaagagagatgaagattcagggactgaagtgggagaaggcacagatgaatgggcccaatccaaagccacagttagaccccctgaccagctggagttgaccgatgcggagttaaaggaggagttcactcggattttgacagccaacaacccacacgcaccccagaacattgtcaggtacagcttcaaagaaggcacatataagcctattggctttgtgaaccaactggcagttcactacacccaggttgggaacctgatccccaaagactcagatgaaggacggcggcagcattaccgcgatgaattagtggcaggttctcaggagtctgtcaaggtgatttcagaaacaggaaacctcgaagaagacgaagagcccaaggagttagaaactgagcctgggagtcaaacagatgtgcctgcagctggggcagctgaaaaagtgactgaagaagaattgatgactcctaagcagcccaaggagagaaagctcactaaccagttcaacttcagtgagagggcctcacagacctacaacaaccctgtccgggatcgagaatgccagacggagcctcctcccaggacaaacttttcagccacagccaatcagtgggagatctatgatgcctatgtagaggaacttgagaagcaggaaaagaccaaagagaaggagaaggcaaagaccccagtggctaaaaaatcagggaagatggccatgaggaagctgacatctatggagtctcagactgatgatctcatcaaattgtcccaagctgctaagatcatggagcggatggtcaaccagaatacatatgatgacattgctcaagattttaagtactatgacgatgctgctgatgaataccgggaccaggtgggtaccctgctgccgctctggaagttccaaaatgacaaagccaagcgcctgtccgtcactgccctctgctggaatccaaagtacagggatctgtttgcagtgggatatggctcttatgacttcatgaagcagagccggggcatgctgctgctctacagcctgaagaaccccagcttccctgagtacatgttcagcagcaacagcggcgtcatgtgtctcgacatccacgtggaccacccctacctggtggcagtaggccactatgacggcaacgtggccatttacaacctcaagaagccccactcccagccctccttctgcagctcagccaagtctggcaagcactcagaccctgtgtggcaggtcaagtggcagaaggatgacatggaccaaaaccttaacttcttctctgtgtcatctgacggcaggattgtgtcttggactctcgtgaagagaaagctggttcacatagatgtcatcaagctgaaggtggaaggcagcaccacggaagttcctgaggggttgcagctgcacccagtgggttgtggcactgcctttgacttccacaaagagattgactacatgttcctagtgggcacagaggagggaaaaatctacaagtgctctaaatcctactccagccaattcctcgacacctatgacgcccacaacatgtcagtggacactgtgtcctggaacccataccacaccaaggtcttcatgtcctgcagctccgactggacagtgaagatctgggaccacaccatcaagaccccgatgttcatctatgacctgaactcagccgtgggtgatgtggcctgggcgccatactcttctactgtgttcgcagcagtcaccacagatgggaaggcccacatatttgacttagccatcaacaagtatgaggccatctgcaaccagcctgtggcggccaaaaagaacaggctcacccacgtgcagttcaatctcatccaccccatcatcattgtgggcgatgaccgtgggcacatcatcagcctcaagctctcacccaatttgcgcaagatgccaaaggaaaagaaggggcaggaggtgcagaagggtccagctgtggagattgcgaaactggacaaactgctgaacctggtgagggaagtgaaaatcaagacctga

SEQ ID NO: 17 - DNA, 2100 bp, ARTIFICIAL SEQUENCE
  Synthetic Nucleotide
atgatcccagcaagcgccaaggcaccacacaagcagccccacaagcagagcatctccatcggcaggggcacaaggaagagggacgaggatagcggaaccgaagtgggagagggaacagacgagtgggcacagtccaaggcaaccgtgcgcccacctgaccagctggagctgacagatgccgagctgaaggaggagttcaccaggatcctgacagccaacaatccacacgccccccagaacatcgtgcgctactctttcaaggagggcacatataagccaatcggctttgtgaaccagctggccgtgcactatacccaagtgggcaatctgatccccaaggactccgatgagggccggagacagcactacagggacgagctggtggcaggatcccaggagtctgtgaaagtgatctctgagaccggcaatctggaggaggacgaggagccaaaggagctggagaccgagccaggaagccagacagatgtgcctgcagcaggagcagcagagaaggtgaccgaggaggagctgatgacacctaagcagccaaaggagcggaagctgaccaaccagttcaatttttccgagagagcctctcagacatacaacaatccagtgcgggacagagagtgccagaccgagccaccccctagaaccaacttttccgccacagccaatcagtgggagatctacgatgcctatgtggaggagctggagaagcaggagaagaccaaggagaaggagaaggccaagacacccgtggccaagaagtccggcaagatggccatgcggaagctgaccagcatggagtcccagacagacgatctgatcaagctgtctcaggccgccaagatcatggagagaatggtgaaccagaatacctatgacgatatcgcccaggacttcaagtactatgacgatgcagcagacgagtacagggatcaagtgggcacactgctgcctctgtggaagtttcagaacgataaggccaagaggctgagcgtgaccgccctgtgctggaatccaaagtacagggacctgttcgcagtgggatacggatcttatgacttcatgaagcagagcagaggcatgctgctgctgtattccctgaagaacccctctttccctgagtacatgtttagctccaattccggcgtgatgtgcctggacatccacgtggatcacccctacctggtggccgtgggccactatgacggcaacgtggccatctacaatctgaagaagcctcactctcagcccagcttctgttctagcgccaagagcggcaagcactccgatcccgtgtggcaggtgaagtggcagaaggacgatatggaccagaacctgaatttcttttccgtgtcctctgatggcaggatcgtgtcttggaccctggtgaagcgcaagctggtgcacatcgacgtgatcaagctgaaggtggagggcagcaccacagaggtgccagagggactgcagctgcacccagtgggatgcggcacagccttcgactttcacaaggagatcgattatatgttcctggtgggcaccgaggagggcaagatctacaagtgttctaagagctatagctcccagtttctggacacatatgatgcccacaacatgagcgtggataccgtgtcctggaatccttaccacacaaaggtgttcatgagctgctctagcgactggaccgtgaagatctgggatcacaccatcaagacacctatgtttatctatgacctgaactccgccgtgggcgatgtggcatgggcaccatactcctctacagtgttcgcagcagtgaccacagacggcaaggcacacatctttgatctggccatcaacaagtacgaggccatctgtaatcagcccgtggccgccaagaagaacaggctgacccacgtgcagttcaatctgatccaccctatcatcatcgtgggcgacgatcggggccacatcatctctctgaagctgagccccaacctgagaaagatgcctaaggagaagaagggacaggaggtgcagaagggaccagcagtggagatcgcaaagctggacaagctgctgaatctggtgcgcgaggtgaagatcaagacctga